SEQUENCE LISTING (7 sequences)

SEQ ID NO: 1 - protein, 77 aa, Homo sapiens
  ref <308>: DATABASE ACCESSION NUMBER: NCBI/NM_017413
  ref <309>: DATABASE ENTRY DATE: 2014-08-09
  ref <313>: RELEVANT RESIDUES IN SEQ ID NO: (1)..(77)
MNLRLCVQALLLLWLSLTAVCGGSLMPLPDGNGLEDGNVRHLVQPRGSRNGPGPWQGGRRKFRRQRPRLSHKGPMPF

SEQ ID NO: 2 - protein, 12 aa, Homo sapiens
RPRLSHKGPMPF

SEQ ID NO: 3 - protein, 13 aa, Homo sapiens
QRPRLSHKGPMPF

SEQ ID NO: 4 - protein, 13 aa, Homo sapiens
  misc_feature (1)..(1): pyroglutamate
XRPRLSHKGPMPF

SEQ ID NO: 5 - protein, 17 aa, Homo sapiens
KFRRQRPRLSHKGPMPF

SEQ ID NO: 6 - protein, 19 aa, Homo sapiens
RRKFRRQRPRLSHKGPMPF

SEQ ID NO: 7 - protein, 36 aa, Homo sapiens
LVQPRGSRNGPGPWQGGRRKFRRQRPRLSHKGPMPF

(19) United States
(12) Patent Application Publication
(10) Pub. No.: US 2016/0058705 A1
(43) Pub. Date: Mar. 3, 2016

(54) COMPOSITIONS AND METHODS FOR TREATING CARDIOVASCULAR AND PULMONARY DISEASES AND DISORDERS WITH APELIN

(71) Applicants: Jayakumar Rajadas, Cupertino, CA (US); Pilar Ruiz-Lozano, Menlo Park, CA (US); Vahid Serpooshan, San Francisco, CA (US)

(72) Inventors: Jayakumar Rajadas, Cupertino, CA (US); Pilar Ruiz-Lozano, Menlo Park, CA (US); Vahid Serpooshan, San Francisco, CA (US)

(21) Appl. No.: 14/838,942

(22) Filed: Aug. 28, 2015

Related U.S. Application Data
(60) Provisional application No. 62/080,025, filed on Nov. 14, 2014.
(60) Provisional application No. 62/043,307, filed on Aug. 28, 2014.

(51) Int. Cl. A61K 9/127; A61K 45/06; A61K 47/42; A61K 38/17; A61K 38/10

(52) U.S. Cl. CPC A61K 9/1271; A61K 38/1709; A61K 38/10; A61K 47/42; A61K 45/06; A61K 9/1277

(57) ABSTRACT

Compositions and methods for treating cardiovascular and pulmonary diseases and disorders with apelin are disclosed. In particular, the invention relates to formulations comprising apelin encapsulated in liposome nanocarriers conjugated with polyethylene glycol (PEG) and their use in treatment of cardiovascular and pulmonary diseases and disorders. Encapsulation of apelin in PEG-conjugated liposomes significantly enhances efficacy, improves cellular uptake of apelin, and allows for sustained and extended release of apelin under physiological conditions.

### CROSS-REFERENCE TO RELATED APPLICATIONS

This application claims benefit under 35 U.S.C. §119(e) of provisional application 62/043,307, filed Aug. 28, 2014, and provisional application 62/080,025, filed Nov. 14, 2014, which applications are hereby incorporated by reference in their entireties.

### TECHNICAL FIELD

The present invention pertains generally to compositions and methods for treating cardiovascular and pulmonary diseases and disorders with apelin. In particular, the invention relates to compositions comprising apelin encapsulated in liposome nanocarriers conjugated with polyethylene glycol (PEG) that enhance the stability of apelin and the use of such compositions in treatment of cardiovascular and pulmonary diseases and disorders.

### BACKGROUND

Cardiac hypertrophy is an adaptive response of the heart cells to elevated levels of biomechanical stress imposed by a variety of extrinsic and intrinsic stimuli including pressure or volume overload, familial/genetic cardiomyopathies, or loss of contractile mass from preceding infarction (Frey et al. (2004) Circulation 109:1580-1589; Frey et al. (2003) Annu. Rev. Physiol. 65:45-79; Yoshida et al. (1986) J. Cardiogr. 16:399-406). If sustained, hypertrophy often becomes pathological, accompanied by significant risk of arrhythmia, progression to heart failure, and sudden death (Frey et al. (2004), supra; Levy et al. (1990) N. Engl. J. Med. 322:1561-1566; Koren et al. (1991) Ann. Intern. Med. 114:345-352). At the molecular level, pathological hypertrophy is associated with re-induction of the so-called fetal gene program in which the fetal isoforms of genes responsible for regulating cardiac contractility and calcium handling (e.g. β-MHC) are upregulated (Frey et al. (2004), supra; Frey et al. (2003), supra); Olson (2004) Nat. Med. 10:467-474; Iemitsu et al. (2001) Am. J. Physiol. Regul. Integr. Comp. Physiol. 281:R2029-2036). At the cellular level, the main characteristics of ventricular hypertrophic growth are enhanced protein synthesis and an increase in size of cardiomyocytes (Frey et al. (2004), supra; Frey et al. (2003), supra). As pathologic hypertrophy progresses, these changes in molecular and cellular phenotypes are accompanied by an increase in apoptosis, fibrosis, chamber dilation, and decreased systolic function (Frey et al. (2004), supra).

The murine model of transverse aortic constriction (TAC) is one of the most common experimental models used to study pressure overload-induced ventricular hypertrophy and elucidate the key signaling processes involved in the cardiac hypertrophic response and its progress to heart failure (DeAlmeida et al. (2010) J. Vis. Exp. April 21; (38) pii:1729; Van Nierop et al. (2013) PLoS ONE 8:e55424; Patten et al. (2009) Circ. Heart Fail. 2:138-144; Rockman et al. (1991) Proc. Natl. Acad. Sci. U.S.A. 88:8277-8281). TAC initially results in compensated hypertrophy, frequently associated with a transient improvement in myocardial contractility; however, chronic hemodynamic overload leads to maladaptive hypertrophy accompanied by ventricular dilatation and heart failure (DeAlmeida et al., supra; Patten et al., supra; Liao et al. (2002) Am. J. Physiol. Heart Circ. Physiol.; 282:H1703-1708; Nakamura et al. (2001) Am. J. Physiol. Heart Circ. Physiol. 281:H1104-1112). The TAC model has been utilized as a platform for examining the utility of pharmacological or molecular interventions that may limit hypertrophy or attenuate the hypertrophy-induced cardiac dysfunction (DeAlmeida et al., supra; Patten et al., supra).

Recent studies have shown that exogenous apelin plays a critical role in ameliorating cardiac dysfunction and/or remodeling in various animal models of cardiac disease such as myocardial infarction, ischemia reperfusion, and hypertrophy (e.g. (Koguchi et al. (2012) Circ. J. Off. J. Jpn. Circ. Soc. 76:137-144; Zhang et al. (2014) Heart Vessels 29(5):679-689; Simpkin et al. (2007) Basic Res. Cardiol. 102:518-528). Apelin is an endogenous peptide ligand for the G-protein-coupled apelin receptor (ApinR, also known as APJ), known to be involved in a broad range of physiological functions, including maintaining body fluid homeostasis, blood pressure, obesity, and heart development and function (Lee et al. (2000) J. Neurochem. 74:34-41; Szokodi et al. (2002) Circ. Res. 91:434-440; El Messari et al. (2004) J. Neurochem. 90:1290-1301; Kuba et al. (2007) Circ. Res. 101:e32-42). Although ApinR and its recently identified ligand, apelin, show high levels of mRNA expression in the heart, their functional significance in the cardiovascular system is not yet fully understood (Szokodi et al., supra; Ashley et al. (2005) Cardiovasc. Res. 65:73-82). Apelin is synthesized as a 77-amino acid peptide processed into various C-terminal fragments, including: apelin-36, apelin-19, apelin-17, apelin-13, and [Pyr1]-apelin-13 (Maguire et al. (2009) Hypertension 54:598-604; Wang et al. (2013) J. Am. Heart Assoc. 2:e000249). [Pyr1]apelin-13 has been recognized as the predominant isoform of apelin in human plasma and cardiac tissue (Maguire et al., supra; Tatemoto et al. (1998) Biochem. Biophys. Res. Commun. 251:471-476; Murza et al. (2014) Biopolymers 101(4):297-303; Azizi et al. (2013) Peptides 46:76-82).

Extensive clinical use of [Pyr1]-apelin-13 has been seriously hampered due to the unstable nature of the peptide in both in vitro and in vivo conditions (Tamargo et al. (2011) Discov. Med. 12:381-392; Andersen et al. (2011) Pulm. Circ. 1:334-346). The instability of apelin in plasma is due to the rapid degradation of the peptide (Maguire et al., supra; Murza et al., supra; Azizi et al., supra; Mesmin et al. (2012) Bioanalysis 4:2851-2863), which results in a significantly short plasma half-life (less than 8 minutes) (Japp et al. (2008) J. Am. Coll. Cardiol. 52:908-913). Thus, there remains a need for improved methods of delivering apelin that would improve stability and therapeutic efficacy.

### SUMMARY OF THE INVENTION

The present invention provides compositions and methods for treating cardiovascular and pulmonary diseases and disorders with apelin. In particular, the invention relates to formulations comprising apelin encapsulated in liposome nanocarriers conjugated with polyethylene glycol (PEG) that enhance the stability of apelin and their use in treatment of cardiovascular and pulmonary diseases and disorders.

In one aspect, the invention includes a composition comprising an apelin peptide encapsulated in a PEG-conjugated liposome. The liposome may comprise lipids selected from the group consisting of 1,2-distearoyl-sn-glycero-3-phosphoethanolamine-N-[amino-(polyethylene glycol)-3400] (DSPE-PEG(3400)-NH₂), cholesterol, and 1,2-distearoyl-sn-glycero-3-phosphocholine (DSPC). In certain embodiments, the composition may further comprise additional agents, for example, that stabilize the apelin peptide and/or affect liposome particle size. In one embodiment, the liposome further comprises bovine serum albumin. The composition may also further comprise a pharmaceutically acceptable excipient. In certain embodiments, the liposome further comprises one or more other drugs for treating a cardiovascular or pulmonary disease or disorder, such as, but not limited to, a vasodilator, an endothelin receptor antagonist, a calcium channel blocker, an anticoagulant, a diuretic, an angiotensin-converting enzyme (ACE) inhibitor, an angiotensin II receptor blocker, a beta blocker, an antiplatelet agent, a cholesterol-lowering drug, and a bronchodilator. Liposomes typically range in size from about 57 nm to about 0.22 μm in diameter depending on the particular apelin peptide, lipid composition, and other components included in the liposome.

In certain embodiments, the apelin peptide comprises an amino acid sequence selected from the group consisting of SEQ ID NOS:1-7 or a sequence displaying at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity thereto. In one embodiment, the apelin peptide is pyroglutamyl apelin-13.

In another aspect, the invention includes a method of treating a subject for a cardiovascular or pulmonary disease or disorder, the method comprising administering a therapeutically effective amount of a composition comprising an apelin peptide encapsulated in a PEG-conjugated liposome to the subject.

Cardiovascular and pulmonary diseases and disorders that can be treated by the methods of the invention include, but are not limited to, cardiac or pulmonary fibrosis, cardiac hypertrophy, aortic aneurism, pulmonary hypertension, hypertensive heart disease, arterial inflammation, inflammatory heart disease, endocarditis, inflammatory cardiomegaly, myocarditis, valvular heart disease, cerebrovascular disease, angina, peripheral and coronary artery disease, heart failure, cor pulmonale, myocarditis, cardiomyopathy, atherosclerosis, congenital heart disease, rheumatic heart disease, myocardial infarction, ischemia-reperfusion injury, cardiac dysrhythmia, inflammatory vascular injury, atrial fibrillation, ventricular tachycardia, Brugada syndrome, preeclampsia, angioplasty, restenosis, vascular surgery, cardiac surgery, and cardiac transplantation.

By “therapeutically effective dose or amount” of a composition comprising apelin encapsulated in liposomes is intended an amount that, when administered as described herein, brings about a positive therapeutic response, such as improved recovery from a cardiovascular or pulmonary disease or disorder. Improved recovery may include improved cardiac repair, increased cardiac contractility, increased cardiac output, reduced pressure overload-induced cardiac dysfunction, decreased fibrosis or hypertrophy, decreased infarct size, decreased inflammation, or decreased mortality. Additionally, a therapeutically effective dose or amount may result in clinical improvement in a patient having a cardiovascular or pulmonary disease or disorder as evidenced, for example, by increased exercise tolerance/capacity, decreased fluid retention, decreased dyspnea, and/or improved results on quantitative tests of cardiac function (e.g., ejection fraction, exercise capacity).

In certain embodiments, multiple therapeutically effective doses of compositions comprising apelin encapsulated in liposomes are administered to the subject. In one embodiment, the composition is administered intermittently, for example, once or twice weekly or every other week.

Any appropriate mode of administration may be used. In certain embodiments, the composition is administered intravenously, subcutaneously, intralesionally, or intraperitoneally. In other embodiments, the composition is administered locally into the heart or vascular system.

In certain embodiments, the method further comprises treating the subject with one or more other drugs or agents for treating a cardiovascular or pulmonary disease or disorder, such as, but not limited to, a vasodilator, an endothelin receptor antagonist, a calcium channel blocker, an anticoagulant, a diuretic, an angiotensin-converting enzyme (ACE) inhibitor, an angiotensin II receptor blocker, a beta blocker, an antiplatelet agent, a cholesterol-lowering drug, a bronchodilator, and oxygen.

In one embodiment, the subject is human. In certain embodiments, the subject has hypertrophy, fibrosis, hypertension, heart failure, cardiomyopathy, atherosclerosis, aortic aneurism, myocardial reperfusion injury, or infarction.

In another aspect, the invention includes a method of preparing a composition comprising an apelin peptide encapsulated in a PEG-conjugated liposome. In one embodiment, the method comprises: a) dissolving cholesterol, NH₂—PEG-DSPE and DSPC in a solvent; b) evaporating the solvent to produce a thin lipid layer; c) adding water to the lipid layer; d) sonicating to produce a turbid liposome solution; e) lyophilizing the liposome solution to produce dry liposomes; and f) encapsulating apelin in the liposomes. Encapsulating apelin in the liposomes may be performed by adding an aqueous solution comprising apelin to the dry liposomes to produce a liposome solution and sonicating the liposome solution. In order to increase the number of liposomes comprising encapsulated apelin, the liposome solution containing apelin may be lyophilized to produce dry liposomes and then rehydrated and sonicated (see Example 1). This procedure may be performed repeatedly until the desired numbers of liposomes comprising encapsulated apelin are produced.

In another aspect, the invention includes a pharmaceutical composition comprising an apelin peptide encapsulated in a PEG-conjugated liposome prepared according to a method described herein.

In another aspect, the invention includes a kit comprising a composition comprising an apelin peptide encapsulated in a PEG-conjugated liposome and instructions for treating a cardiovascular or pulmonary disease or disorder. The composition in the kit may further comprise a pharmaceutically acceptable excipient. The kit may also further comprise one or more other drugs for treating a cardiovascular or pulmonary disease or disorder. Additionally, the kit may further comprise means for delivering the composition to a subject.

These and other embodiments of the subject invention will readily occur to those of skill in the art in view of the disclosure herein.

### BRIEF DESCRIPTION OF THE DRAWINGS

FIGS. 1A-1C show schematic representations and atomic force microscopy (AFM) images of empty PEG liposomes and PEG liposomes with [Pyr1]-apelin-13 encapsulated. FIG. 1A shows schematic representations of empty PEG liposomes and PEG liposomes with [Pyr1]-apelin-13 encapsulated. AFM images of empty PEG liposomes (FIG. 1B) and PEG liposomes with [Pyr1]-apelin-13 encapsulated (FIG. 1C) demonstrate the morphology of the nanocarrier systems used in this study.

FIG. 2A shows DLS data demonstrating the liposome particle sizes. Error bars indicate standard deviations of liposome sizes based on cumulative analysis. FIG. 2B and FIG. 2C show time courses of [Pyr1]-apelin-13 release from liposomes in the absence (FIG. 2B) and presence (FIG. 2C) of 0.5% bovine serum albumin (BSA), by rapid equilibrium dialysis plate assay. The quantity (μg/mL) of [Pyr1]-apelin-13 released from the donor to buffer reservoir over a 24 hour time course at various intervals is indicated.

FIGS. 3A-3F show in vitro assessment of the effect of the [Pyr1]-apelin-13 nanocarriers on APJ translocation (white arrows) in HEK APJ-GFP+ cells (t=60 minutes). FIG. 3A and FIG. 3B show negative controls, without [Pyr1]-apelin-13. FIG. 3C and FIG. 3D show the regular [Pyr1]-apelin-13 effect. FIG. 3E and FIG. 3F show the effect of addition of lipoPEG-[Pyr1]-apelin-13 nanocarriers. The nuclei were stained with 4′,6-diamidino-2-phenylindole (DAPI).

FIGS. 4A and 4B show a schematic representation of the experimental procedure used to assess the effect of administration of lipoPEG-[Pyr1]-apelin-13 nanocarriers in vivo on the transverse aortic constriction (TAC)-induced cardiac hypertrophy in mouse. FIG. 4A shows the time line for each step of the procedure. FIG. 4B illustrates the method of performing TAC to induce cardiac hypertrophy, lipoPEG-[Pyr1]-apelin-13 administration, and assessment of the effects of lipoPEG-[Pyr1]-apelin-13 administration by histology.

FIGS. 5A-5E show the percentage changes in echocardiographic parameters compared to baseline (divided to the pre-surgery values), at days 1 (FIG. 5A), 8 (FIG. 5B), and 15 (FIG. 5C) post-surgery (TAC). Moreover, FIG. 5D demonstrates the absolute fractional shortening (FS) values for the four different groups as a function of time post-surgery. Saline (sham controls), [Pyr1]-apelin-13, or lipoPEG-[Pyr1]-apelin-13 (lipoPEG-PA13) were injected IP (300 μg/Kg body weight) at days 1 and 7 post surgery. While sham controls, either injected with saline (not shown) or lipoPEG-PA13 nanocarriers ▪, showed no significant changes in the heart function over 2-week time period, TAC hearts with saline treatment ▪ showed significant decline in cardiac contractility or fractional shortening (FS) and an increase in left ventricle (LV) internal diameter (LVID) as well as LV posterior wall thickness (LVPW), indicating a hypertrophic response and cardiac dysfunction. Administration of commercial [Pyr1]-apelin-13 ▪ resulted in slight inhibition of cardiac remodeling and increase in FS, when compared with TAC controls. The greater effect however, was observed in the group treated with lipoPEG-PA13 carriers ▪ in which cardiac contractility (FS) was significantly improved compared with those in other TAC groups (FIG. 5D), and hypertrophic remodeling was significantly inhibited. As shown in panel (FIG. 5D), the four animal groups of this study showed varying levels of FS at the baseline. Considering these differences, the significant effect of nanocarrier treatment in improving FS is evident. A minimum of four replicates were analyzed for each group. Statistical analysis was performed using one-way ANOVA test. Values are reported as average±SEM. *: p<0.05 compared to sham controls (▪). ▴: p<0.05 compared to TAC injected with [Pyr1]-apelin-13 (▪). FIG. 5E shows representative heart sections with Masson's trichrome staining for all experimental groups. While TAC caused a remarkable left ventricular hypertrophic growth along with a significant amount of fibrotic tissue in untreated TAC hearts, administration of [Pyr1]-apelin-13 or lipoPEG-PA13 nanocarriers resulted in remarkable inhibition of the LV growth, attenuating TAC-induced cardiac dysfunction, and diminishing fibrosis. Scale bar is 2 mm.

FIGS. 6A and 6B show [Pyr1]-apelin-13 release in vivo. The [Pyr1]-apelin-13 concentration in blood plasma was measured by ELISA at 1, 4, and 6 days post IP injection. FIG. 6A shows sham mice injected with saline (control ▪) or lipoPEG-[Pyr1]-apelin-13 (lipoPEG-PA13) (▪). FIG. 6B shows TAC mice injected with saline (control ≡) or lipoPEG-PA13 nanocarriers (▪). These were compared with [Pyr1]-apelin-13 concentration in blood plasma of wild type (WT) mice with no injury and no injections (▪). In both sham and TAC animals, using the nanocarriers resulted in significantly elevated levels of [Pyr1]-apelin-13 in blood plasma for up to 6 days post injection, compared to those in saline-injected animals as well as those in control WT mice. A minimum of four replicates were analyzed for each group. Statistical analysis was performed using a one-way ANOVA test. Values are reported as average±SEM.+: p<0.05 compared to WT controls. *: p<0.05 compared to same time, injected with saline. ▪: p<0.05 compared to sham at the same time, injected with lipoPEG-PA13 carriers.

### DETAILED DESCRIPTION OF THE INVENTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., R. O. Bonow, D. L. Mann, D. P. Zipes, P. Libby Braunwald's Heart Disease: A Textbook of Cardiovascular Medicine (Saunders; 9^{th} edition, 2011); J. Watchie Cardiovascular and Pulmonary Physical Therapy: A Clinical Manual (Saunders, 2^{nd} edition, 2009); R. A. Walsh Molecular Mechanisms of Cardiac Hypertrophy and Failure (CRC Press, 2004); Genetics of Cardiovascular Disease, Volume 124 (Progress in Molecular Biology and Translational Science, T. Chico ed., Academic Press, 2014); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook et al., Molecular Cloning: A Laboratory Manual (Cold Spring Harbor Laboratory Press, 3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a liposome” includes a mixture of two or more such liposomes, and the like.

The term “about,” particularly in reference to a given quantity, is meant to encompass deviations of plus or minus five percent.

The term “apelin” as used herein encompasses all forms of apelin, including the mature forms of apelin, such as apelin-36, apelin-19, apelin-17, apelin-13, [Pyr1]-apelin-13, and apelin-12, as well as the immature forms of apelin, such as prepro-apelin and pro-apelin, and also includes biologically active fragments, variants, analogs, and derivatives thereof that retain apelin biological activity (e.g., interact with and regulate the apelin receptor).

An apelin polynucleotide, nucleic acid, oligonucleotide, protein, polypeptide, or peptide refers to a molecule derived from any source. The molecule need not be physically derived from an organism, but may be synthetically or recombinantly produced. Apelin sequences from a number of species are well known in the art and include, but are not limited to, the following: Homo sapiens apelin (GenBank Accession No. 290645488), Rattus norvegicus apelin (GenBank Accession No. 398650649); Bos taurus apelin (GenBank Accession No. 27806214), Mus musculus apelin (GenBank Accession No. 123702126), Danio rerio apelin (GenBank Accession No. 261244911), Astyanax mexicanus apelin (GenBank Accession No. 584299653), Carassius auratus apelin (GenBank Accession No. 254654106), Tautogolabrus adspersus apelin (GenBank Accession No. 584012919), Sus scrofa apelin (GenBank Accession No. 209978363), Pan troglodytes apelin (GenBank Accession No. 410329960), and Xenopus laevis clone XL005a15 preproapelin-b (GenBank Accession No. 93278258); all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to produce a composition comprising apelin encapsulated in liposomes as described herein.

The terms “peptide,” “oligopeptide,” and “polypeptide” refer to any compound comprising naturally occurring or synthetic amino acid polymers or amino acid-like molecules including but not limited to compounds comprising amino and/or imino molecules. No particular size is implied by use of the terms “peptide,” “oligopeptide” or “polypeptide” and these terms are used interchangeably. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, linearly-linked peptides), cyclized, branched molecules and the like, are included within the definition. The terms also include molecules comprising one or more peptoids (e.g., N-substituted glycine residues) and other synthetic amino acids or peptides. (See, e.g., U.S. Pat. Nos. 5,831,005; 5,877,278; and 5,977,301; Nguyen et al. (2000) Chem Biol. 7(7):463-473; and Simon et al. (1992) Proc. Natl. Acad. Sci. USA 89(20):9367-9371 for descriptions of peptoids). Further, peptides and polypeptides, as described herein, for example synthetic peptides, may include additional molecules such as labels or other chemical moieties. Such moieties may further enhance biological activity (e.g., interaction of the apelin peptides with the apelin receptor), and/or stability of the apelin peptides, and/or further detection of the apelin peptides.

Thus, references to polypeptides or peptides also include derivatives of the amino acid sequences of the invention including one or more non-naturally occurring amino acids. A first polypeptide or peptide is “derived from” a second polypeptide or peptide if it is (i) encoded by a first polynucleotide derived from a second polynucleotide encoding the second polypeptide or peptide, or (ii) displays sequence identity to the second polypeptide or peptide as described herein. Sequence (or percent) identity can be determined as described below. Preferably, derivatives exhibit at least about 50% percent identity, more preferably at least about 80%, and even more preferably between about 85% and 99% (or any value therebetween) to the sequence from which they were derived. Such derivatives can include postexpression modifications of the polypeptide or peptide, for example, pyroglutamylation, glycosylation, acetylation, methylation, phosphorylation, lipidation, and the like.

Amino acid derivatives can also include modifications to the native sequence, such as deletions, additions and substitutions (generally conservative in nature), so long as the polypeptide or peptide maintains the desired activity (e.g., apelin biological activity). These modifications may be deliberate, as through site-directed mutagenesis, or may be accidental, such as through mutations of hosts that produce the proteins or errors due to PCR amplification. Furthermore, modifications may be made that have one or more of the following effects: increasing affinity and/or specificity for the apelin receptor and facilitating cell processing. Polypeptides and peptides described herein can be made recombinantly, synthetically, or in tissue culture.

The term “derived from” is used herein to identify the original source of a molecule but is not meant to limit the method by which the molecule is made which can be, for example, by chemical synthesis or recombinant means.

The terms “variant,” “analog” and “mutein” refer to biologically active derivatives of the reference molecule that retain desired activity, such as apelin activity for use in the treatment of a cardiovascular or pulmonary disease or disorder as described herein. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem Biol. (2000) 7:463-473; and Simon et al., Proc. Natl. Acad. Sci. USA (1992) 89:9367-9371 for descriptions of peptoids). Preferably, the analog or mutein has at least the same apelin biological activity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

By “fragment” is intended a molecule consisting of only a part of the intact full-length sequence and structure. The fragment can include a C-terminal deletion an N-terminal deletion, and/or an internal deletion of the native polypeptide. Active fragments of a particular protein will generally include at least about 5-10 contiguous amino acid residues of the full-length molecule, but may include at least about 10-36 contiguous amino acid residues of the full-length molecule, or at least about 20-50 or more contiguous amino acid residues of the full-length molecule, or any integer between 5 amino acids and the full-length sequence, provided that the fragment in question retains biological activity, such as apelin activity, as defined herein.

“Substantially purified” generally refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

“Cardiovascular and pulmonary diseases and disorders” include, but are not limited to, cardiac or pulmonary fibrosis, cardiac hypertrophy, aortic aneurism, pulmonary hypertension, hypertensive heart disease, arterial inflammation, inflammatory heart disease, endocarditis, inflammatory cardiomegaly, myocarditis, valvular heart disease, cerebrovascular disease, angina, peripheral and coronary artery disease, heart failure, cor pulmonale, myocarditis, cardiomyopathy, atherosclerosis, congenital heart disease, rheumatic heart disease, myocardial infarction, ischemia-reperfusion injury, cardiac dysrhythmia, inflammatory vascular injury, atrial fibrillation, ventricular tachycardia, Brugada syndrome, preeclampsia, angioplasty, restenosis, vascular surgery, cardiac surgery, and cardiac transplantation.

The term “treatment” or “treating” as used herein refers to the ability to ameliorate, suppress, mitigate, or eliminate the clinical symptoms of a cardiovascular or pulmonary disease or disorder.

An “effective amount” of a composition comprising apelin encapsulated in liposomes is an amount sufficient to effect beneficial or desired results, such as an amount that activates the apelin receptor and/or induces vasodilatation, angiogenesis, or proliferation of myocardial progenitor cells. An effective amount can be administered in one or more administrations, applications or dosages.

By “therapeutically effective dose or amount” of a composition comprising apelin encapsulated in liposomes is intended an amount that, when administered as described herein, brings about a positive therapeutic response, such as improved recovery from a cardiovascular or pulmonary disease or disorder. Improved recovery may include improved cardiac repair, increased cardiac contractility, increased cardiac output, reduced pressure overload-induced cardiac dysfunction, decreased fibrosis or hypertrophy, decreased infarct size, decreased inflammation, or decreased mortality. Additionally, a therapeutically effective dose or amount may result in clinical improvement in a patient having a cardiovascular or pulmonary disease or disorder as evidenced, for example, by increased exercise tolerance/capacity, decreased fluid retention, decreased dyspnea, and/or improved results on quantitative tests of cardiac function (e.g., ejection fraction, exercise capacity). The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of a novel drug delivery system for apelin comprising PEG-conjugated liposomal nanoparticles. Apelin is an adipokine that regulates a variety of biological functions including cardiac hypertrophy and hypertrophy-induced heart failure. The clinical use of apelin has been greatly impaired by its short half-life in circulation in the bloodstream. The inventors have shown that encapsulation of [Pyr1]-apelin-13 in liposome nanocarriers, conjugated with PEG polymer on their surface, prolongs apelin stability in the bloodstream and results in sustained and extended drug release both in vitro and in vivo. Moreover, intraperitoneal injection of [Pyr1]-apelin-13 nanocarriers in a mouse model of pressure-overload induced heart failure demonstrated a sustainable long-term effect of [Pyr1]-apelin-13 in preventing cardiac dysfunction.

In order to further an understanding of the invention, a more detailed discussion is provided below regarding apelin, as well as modes of delivery of apelin encapsulated in liposomes and methods of using apelin for treatment of cardiovascular and pulmonary diseases and disorders.

A. Apelin

As explained above, the methods of the present invention include administering apelin encapsulated in liposomes. Any form of apelin may be used in the practice of the invention, including mature forms of apelin, such as apelin-36, apelin-19, apelin-17, apelin-13, [Pyr1]-apelin-13, and apelin-12, and immature forms of apelin, such as prepro-apelin and pro-apelin, as well as biologically active fragments, variants, analogs, and derivatives thereof that retain apelin biological activity (e.g., interact with and regulate the apelin receptor, promote angiogenesis, or induce proliferation of myocardial progenitor cells).

The apelin for use in the methods of the invention may be native, obtained by recombinant techniques, or produced synthetically, and may be from any source. Representative human apelin sequences are presented in SEQ ID NOS:1-7 for the preproprotein form and various isoforms of apelin (e.g., apelin-12, apelin-13, pyroglutamyl apelin-13, apelin 17, apelin-19, and apelin 36). In addition, apelin sequences from a number of species are well known in the art and include but are not limited to, the following: Homo sapiens apelin (GenBank Accession No. 290645488), Rattus norvegicus apelin (GenBank Accession No. 398650649); Bos taurus apelin (GenBank Accession No. 27806214), Mus musculus apelin (GenBank Accession No. 123702126), Danio rerio apelin (GenBank Accession No. 261244911), Astyanax mexicanus apelin (GenBank Accession No. 584299653), Carassius auratus apelin (GenBank Accession No. 254654106), Tautogolabrus adspersus apelin (GenBank Accession No. 584012919), Sus scrofa apelin (GenBank Accession No. 209978363), Pan troglodytes apelin (GenBank Accession No. 410329960), and Xenopus laevis clone XL005a15 preproapelin-b (GenBank Accession No. 93278258); all of which sequences (as entered by the date of filing of this application) are herein incorporated by reference. Any of these sequences, or a biologically active fragment thereof, or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to produce a composition comprising apelin encapsulated in liposomes as described herein. Although any source of apelin can be utilized to practice the invention, preferably the apelin is derived from a human source, particularly when the subject undergoing therapy is human.

According to various embodiments of the invention, the complete apelin preproprotein (SEQ ID NO:1) or any biologically active peptide obtained by cleavage of the 77 amino acid preproprotein, may be used in the methods described herein. Biologically active fragments of apelin will generally include at least about 5-77 contiguous amino acid residues of the full length apelin preproprotein, but may include at least about 8-50 contiguous amino acid residues of the full length molecule, and may include at least about 10-36 or more contiguous amino acid residues of the full length molecule, or any integer between 5 amino acids and the full length sequence, provided that the fragment in question retains biological activity, such as the ability to activate the apelin receptor. Additionally, apelin peptides may induce angiogenesis, vasodilation, or proliferation of myocardial progenitor cells, or reduce fibrosis or hypertrophy. In certain embodiments, a peptide selected from the group consisting of apelin-12 (SEQ ID NO:2), apelin-13 (SEQ ID NO:3), pyroglutamyl apelin 13 (SEQ ID NO:4), apelin-17 (SEQ ID NO:5), apelin-19 (SEQ ID NO:6), and apelin-36 (SEQ ID NO:7) is used. In other embodiments, a biologically active fragment of apelin-12 consisting of 6, 7, 8, 9, 10, or 11 contiguous amino acids of apelin-12 is used.

The compositions useful in the methods of the invention may comprise biologically active variants of apelin, including variants of apelin from any species. Such variants should retain the desired biological activity of the native peptide such that the pharmaceutical composition comprising the variant peptide has the same therapeutic effect as the pharmaceutical composition comprising the native peptide when administered to a subject. That is, the variant peptide will serve as a therapeutically active component in the pharmaceutical composition in a manner similar to that observed for the native peptide. Methods are available in the art for determining whether a variant peptide retains the desired biological activity, and hence serves as a therapeutically active component in the pharmaceutical composition. Biological activity can be measured using assays specifically designed for measuring activity of the native peptide, including assays described herein (see Example 1). Additionally, antibodies raised against a biologically active native peptide can be tested for their ability to bind to a variant peptide, where effective binding is indicative of a peptide having a conformation similar to that of the native peptide.

Suitable biologically active variants of native or naturally occurring apelin can be biologically active fragments, analogs, muteins, and derivatives of the peptide, as defined above. For example, amino acid sequence variants of the peptide can be prepared by introducing mutations in the cloned DNA sequence encoding the native peptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382); Sambrook et al. (2001) Molecular Cloning: A Laboratory Manual (Cold Spring Harbor Laboratory Press, 3^{rd} Edition); U.S. Pat. No. 4,873,192; and the references cited therein; herein incorporated by reference. Guidance as to appropriate amino acid substitutions that do not destroy biological activity of a peptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

Guidance as to the regions of apelin that can be altered by residue substitutions, deletions, or insertions can be found in the art. See, for example, the structure/function relationships and/or binding studies discussed in Zhang et al. (2014) Bioorg. Med. Chem. 22(11):2992-2997, Langelaan et al. (2013) Biochim. Biophys. Acta 1828(6):1471-1483, Charles (2011) Expert Opin. Drug Discov. 6(6):633-644, Murza et al. (2012) ChemMedChem. 7(2):318-325, Langelaan et al. (2009) Biochemistry 48(3):537-548, Zhou et al. (2003) Virology 317(1):84-94, Fan et al. (2003) Biochemistry 42(34):10163-10168; the contents of which are herein incorporated by reference in their entireties.

In constructing variants of apelin, modifications are made such that variants continue to possess the desired activity. Obviously, any mutations made in the DNA encoding the variant polypeptide must not place the sequence out of reading frame and preferably will not create complementary regions that could produce secondary mRNA structure.

Biologically active variants of apelin will generally have at least about 70%, preferably at least about 80%, more preferably at least about 90% to 95% or more, and most preferably at least about 98%, 99%, or more amino acid sequence identity to the amino acid sequence of a reference apelin peptide molecule (e.g., native preproapelin (SEQ ID NO:1), apelin-12 (SEQ ID NO:2), apelin-13 (SEQ ID NO:3), pyroglutamyl apelin 13 (SEQ ID NO:4), apelin-17 (SEQ ID NO:5), apelin-19 (SEQ ID NO:6), or apelin-36 (SEQ ID NO:7)), which serves as the basis for comparison. A variant may, for example, differ by as few as 1 to 15 amino acid residues, as few as 1 to 10 residues, such as 6-10, as few as 5, as few as 4, 3, 2, or even 1 amino acid residue.

With respect to optimal alignment of two amino acid sequences, the contiguous segment of the variant amino acid sequence may have the same number of amino acids, additional amino acid residues or deleted amino acid residues with respect to the reference amino acid sequence. The contiguous segment used for comparison to the reference amino acid sequence will typically include at least 8 contiguous amino acid residues, and may be 10, 12, 13, 17, 36, 40, 50, 60, 70, or more amino acid residues. Corrections for sequence identity associated with conservative residue substitutions or gaps can be made (see, e.g., Smith-Waterman homology search algorithm). A biologically active variant of a native apelin peptide of interest may differ from the native peptide by as few as 1-20 amino acids, including as few as 1-15, as few as 1-10, such as 6-10, or as few as 5, including as few as 4, 3, 2, or even 1 amino acid residue.

The precise chemical structure of a peptide having apelin activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of peptides having apelin activity as used herein. Further, the primary amino acid sequence of the peptide may be augmented by derivatization using sugar moieties (glycosylation), polyethylene glycol (PEG), or by other supplementary molecules such as lipids, phosphate, acetyl, methyl, or pyroglutamyl groups, and the like. It may also be augmented by conjugation with saccharides. Certain aspects of such augmentation are accomplished through post-translational processing systems of the producing host; other such modifications may be introduced in vitro. In any event, such modifications are included in the definition of an apelin peptide used herein as long as the apelin activity of the peptide is not destroyed. It is expected that such modifications may quantitatively or qualitatively affect the activity, either by enhancing or diminishing the activity of the peptide, in the various assays. Further, individual amino acid residues in the chain may be modified by oxidation, reduction, or other derivatization, and the peptide may be cleaved to obtain fragments that retain activity. Such alterations that do not destroy activity do not remove the peptide sequence from the definition of apelin peptides of interest as used herein.

The art provides substantial guidance regarding the preparation and use of apelin variants. In preparing apelin variants, one of skill in the art can readily determine which modifications to the native apelin nucleotide or amino acid sequence will result in a variant that is suitable for use as a therapeutically active component of a pharmaceutical composition used in the methods of the present invention. For examples of apelin variants, see, e.g., Pisarenko et al. (2014) Mol. Cell. Biochem. 391(1-2):241-250, Pisarenko et al. (2013) J. Pharmacol. Pharmacother. 4(3):198-203, Wang et al. (2013) J. Am. Heart Assoc. 2(4):e000249, Cayabyab et al. (2000) J. Viol. 74(24):11972-11976, Zhang et al. (2014) Bioorg Med Chem. 22(11):2992-2997, Murza et al. (2012) ChemMedChem. 7(2):318-325, and U.S. patent application publications 20140142022 and 20140142049; herein incorporated by reference in their entireties. In addition, a number of apelin variants are also commercially available, including mutated and/or lipidated apelin-12 and apelin-13 analogs from Innovagen (Lund, Sweden) and Rockland Immunochemicals Inc. (Gilbertsville, Pa.).

B. Production of Apelin Peptides

Apelin peptides can be prepared in any suitable manner (e.g., recombinant expression, purification from cell culture, chemical synthesis, etc.) and in various forms (e.g. native, mutated, pyroglutamylated, lipidated, fusions, labeled, etc.). Apelin peptides include naturally-occurring peptides, recombinantly produced peptides, synthetically produced peptides, or peptides produced by a combination of these methods. Means for preparing peptides are well understood in the art. Peptides are preferably prepared in substantially pure form (i.e. substantially free from other host cell or non-host cell proteins).

In one embodiment, the peptides are generated using recombinant techniques. One of skill in the art can readily determine nucleotide sequences that encode the desired peptides using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding peptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding peptides that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified peptides can be generated by creating expression constructs which operably link, in various combinations, polynucleotides encoding peptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the apelin peptides described herein. Generally, such systems use virus-based vectors to transfect plant cells with heterologous genes. For a description of such systems, see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired peptide is transcribed into RNA in the host cell transformed by a vector containing this expression construction. The coding sequence may or may not contain a signal peptide or leader sequence. With the present invention, both the naturally occurring signal peptides and heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorphs, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the fusion proteins of the present invention are produced by growing host cells transformed by an expression vector described above under conditions whereby the protein of interest is expressed. The selection of the appropriate growth conditions is within the skill of the art.

In one embodiment, the transformed cells secrete the peptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted peptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant peptides or polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced peptides or polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular peptides or polypeptides of the present invention involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the peptides or polypeptides can be further purified using conventional techniques well known in the art, such as by any of the techniques described above.

Apelin peptides can be conveniently synthesized chemically, for example by any of several techniques that are known to those skilled in the peptide art. See, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999); herein incorporated by reference.

In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final peptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

Apelin peptides can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

Once an apelin peptide is obtained, the apelin peptide can be encapsulated in a polyethylene glycol (PEG)-conjugated liposome. See, e.g., Example 1 for a description of the preparation of such liposomes using 1,2-distearoyl-sn-glycero-3-phosphoethanolamine-N-[amino-(polyethylene glycol)-3400] (DSPE-PEG(3400)-NH₂), cholesterol, and 1,2-distearoyl-sn-glycero-3-phosphocholine (DSPC). Encapsulation of apelin in PEG-conjugated liposomes significantly enhances efficacy, improves cellular uptake of apelin, and allows for sustained and extended release of apelin under physiological conditions.

C. Pharmaceutical Compositions

Apelin encapsulated in liposomes, as described herein, can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the apelin or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the apelin (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D. C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising apelin encapsulated in liposomes are in unit dosage form, meaning an amount of a composition of the invention appropriate for a single dose, in a premeasured or pre-packaged form.

The compositions herein may optionally include one or more additional agents, such as other drugs for treating a cardiovascular or pulmonary disease or disorder, or other medications used to treat a subject for a condition or disease. Particularly preferred are compounded preparations including apelin and one or more drugs for treating a cardiovascular or pulmonary disease or disorder, such as vasodilators, such as epoprostenol (Flolan), iloprost (Ventavis), isosorbide dinitrate (Isordil), nesiritide (Natrecor), hydralazine (Apresoline), nitrates, and minoxidil, endothelin receptor antagonists, such as bosentan (Tracleer) and Ambrisentan (Letairis), drugs that open the blood vessels in the lungs to improve blood flow, such as sildenafil (Revatio, Viagra) and tadalafil (Cialis, Adcirca), calcium channel blockers, such as amlodipine (Norvasc, Lotrel), bepridil (Vascor), diltiazem (Cardizem, Tiazac), felodipine (Plendil), nifedipine (Adalat, Procardia), nimodipine (Nimotop), nisoldipine (Sular), and verapamil (Calan, Isoptin, Verelan), anticoagulants, such as warfarin (Coumadin, Jantoven), dalteparin (Fragmin), danaparoid (Orgaran), enoxaparin (Lovenox), heparin, and Tinzaparin (Innohep), angiotensin-converting enzyme (ACE) inhibitors, such as benazepril (Lotensin), captopril (Capoten), enalapril (Vasotec), fosinopril (Monopril), lisinopril (Prinivil, Zestril), moexipril (Univasc), perindopril (Aceon), quinapril (Accupril), ramipril (Altace), and trandolapril (Mavik), angiotensin II receptor blockers, such as candesartan (Atacand), eprosartan (Teveten), irbesartan (Avapro), losartan (Cozaar), telmisartan (Micardis), and valsartan (Diovan), beta blockers, such as acebutolol (Sectral), atenolol (Tenormin), betaxolol (Kerlone), bisoprolol/hydrochlorothiazide (Ziac), bisoprolol (Zebeta), carteolol (Cartrol), metoprolol (Lopressor, Toprol XL), nadolol (Corgard), propranolol (Inderal), sotalol (Betapace), timolol (Blocadren), diuretics, such as furosemide (Lasix), bumetanide (Bumex), torsemide (Demadex), hydrochlorothiazide (Esidrix), metolazone (Zaroxolyn), and spironolactone (Aldactone), antiplatelet agents, such as aspirin, ticlopidine, clopidogrel (Plavix), and dipyridamole, cholesterol-lowering drugs, such as statins, resins, nicotinic acid (niacin), gemfibrozil, and clofibrate, and digoxin, and bronchodilators, such as aminophylline, theophylline, salbutamol, salmeterol, bambuterol, clenbuterol, formoterol, indacaterol, tiotropium, and ipratropium bromide. Alternatively, such agents can be contained in a separate composition from the composition comprising apelin and co-administered concurrently, before, or after the composition comprising apelin.

D. Administration

At least one therapeutically effective cycle of treatment with a composition comprising apelin encapsulated in liposomes, as described herein, will be administered to a subject for treatment of a cardiovascular or pulmonary disease or disorder. Cardiovascular and pulmonary diseases and disorders include, but are not limited to, cardiac fibrosis, pulmonary fibrosis, cardiac hypertrophy, aortic aneurism, pulmonary hypertension, hypertensive heart disease, arterial inflammation, inflammatory heart disease, endocarditis, inflammatory cardiomegaly, myocarditis, valvular heart disease, cerebrovascular disease, angina, peripheral and coronary artery disease, heart failure, cor pulmonale, myocarditis, cardiomyopathy, atherosclerosis, congenital heart disease, rheumatic heart disease, myocardial infarction, ischemia-reperfusion injury, cardiac dysrhythmia, inflammatory vascular injury, atrial fibrillation, ventricular tachycardia, Brugada syndrome, preeclampsia, angioplasty, restenosis, vascular surgery, cardiac surgery, and cardiac transplantation.

By “therapeutically effective cycle of treatment” is intended a cycle of treatment that when administered, brings about a positive therapeutic response with respect to treatment of an individual for a cardiovascular or pulmonary disease or disorder. Of particular interest is a cycle of treatment with a composition comprising apelin encapsulated in liposomes that induces vasodilation, angiogenesis, or proliferation of myocardial progenitor cells. By “positive therapeutic response” is intended that the individual undergoing treatment according to the invention exhibits an improvement in one or more symptoms of a cardiovascular or pulmonary disease or disorder, including such improvements as enhanced cardiac repair, increased cardiac contractility, increased cardiac output, reduced pressure overload-induced cardiac dysfunction, decreased fibrosis or hypertrophy, decreased infarct size, decreased inflammation, or decreased mortality. Additionally, a therapeutically effective dose or amount may result in clinical improvement in a patient having a cardiovascular or pulmonary disease or disorder as evidenced, for example, by increased exercise tolerance/capacity, decreased fluid retention, decreased dyspnea, and/or improved results on quantitative tests of cardiac function (e.g., ejection fraction, exercise capacity).

In certain embodiments, multiple therapeutically effective doses of compositions comprising apelin encapsulated in liposomes, and/or one or more other therapeutic agents, such as other apelin receptor agonists or drugs for treating a cardiovascular or pulmonary disease or disorder, or other medications will be administered. The compositions of the present invention are typically, although not necessarily, administered orally, via injection (subcutaneously, intravenously, or intramuscularly), by infusion, or locally. Additional modes of administration are also contemplated, such as intra-arterial, intravascular, pulmonary, intracardiac, intramyocardial, intrapericardial, intraspinal, intralesion, intraparenchymatous, rectal, transdermal, transmucosal, intrathecal, intraocular, intraperitoneal, and so forth. In particular embodiments, compositions are administered into an artery, vein, or capillary of a subject.

The preparations according to the invention are also suitable for local treatment. In a particular embodiment, a composition of the invention is used for localized delivery of a composition comprising apelin encapsulated in liposomes, for example, for the treatment of a cardiovascular or pulmonary disease or disorder. For example, compositions may be administered directly into the heart or vascular system (e.g., arteries, veins, or capillaries). The particular preparation and appropriate method of administration are chosen to target apelin to the site in need of angiogenesis or cardiovascular repair.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising apelin encapsulated in liposomes and/or other agents may be administered using the same or different routes of administration in accordance with any medically acceptable method known in the art.

In another embodiment, the pharmaceutical compositions comprising apelin encapsulated in liposomes and/or other agents are administered prophylactically, e.g., to prevent cardiovascular damage (e.g., hypertrophy or fibrosis) or improve cardiac function. Such prophylactic uses will be of particular value for subjects with congenital heart disease, subjects who are susceptible to cardiac fibrosis or hypertrophy, subjects who are at risk of cardiovascular complications due to obesity, high cholesterol levels, high blood pressure, hyperlipidemia, diabetes mellitus, tobacco smoking, alcoholism, or a surgical procedure, or subjects who have a genetic predisposition to developing a cardiovascular or pulmonary disease or disorder.

In another embodiment of the invention, the pharmaceutical compositions comprising apelin encapsulated in liposomes and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a pharmaceutical composition.

The invention also provides a method for administering a conjugate comprising an apelin peptide encapsulated in liposomes as provided herein to a patient suffering from a condition that is responsive to treatment with apelin contained in the conjugate or composition. The method comprises administering, via any of the herein described modes, a therapeutically effective amount of the conjugate or drug delivery system, preferably provided as part of a pharmaceutical composition. The method of administering may be used to treat any condition that is responsive to treatment with an apelin peptide. More specifically, the compositions herein are effective in treating a cardiovascular or pulmonary disease or disorder.

Those of ordinary skill in the art will appreciate which conditions a specific apelin peptide can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case. In certain embodiments, multiple therapeutically effective doses of each of a composition comprising apelin encapsulated in liposomes will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising apelin encapsulated in liposomes will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the specific apelin peptide, the magnitude of the effect desired, and the route of administration.

Apelin encapsulated in liposomes (again, preferably provided as part of a pharmaceutical preparation) can be administered alone or in combination with one or more other therapeutic agents, such as vasodilators, such as epoprostenol (Flolan), iloprost (Ventavis), isosorbide dinitrate (Isordil), nesiritide (Natrecor), hydralazine (Apresoline), nitrates, and minoxidil, endothelin receptor antagonists, such as bosentan (Tracleer) and Ambrisentan (Letairis), drugs that open the blood vessels in the lungs to improve blood flow, such as sildenafil (Revatio, Viagra) and tadalafil (Cialis, Adcirca), calcium channel blockers, such as amlodipine (Norvasc, Lotrel), bepridil (Vascor), diltiazem (Cardizem, Tiazac), felodipine (Plendil), nifedipine (Adalat, Procardia), nimodipine (Nimotop), nisoldipine (Sular), and verapamil (Calan, Isoptin, Verelan), anticoagulants, such as warfarin (Coumadin, Jantoven), dalteparin (Fragmin), danaparoid (Orgaran), enoxaparin (Lovenox), heparin, and Tinzaparin (Innohep), angiotensin-converting enzyme (ACE) inhibitors, such as benazepril (Lotensin), captopril (Capoten), enalapril (Vasotec), fosinopril (Monopril), lisinopril (Prinivil, Zestril), moexipril (Univasc), perindopril (Aceon), quinapril (Accupril), ramipril (Altace), and trandolapril (Mavik), angiotensin II receptor blockers, such as candesartan (Atacand), eprosartan (Teveten), irbesartan (Avapro), losartan (Cozaar), telmisartan (Micardis), and valsartan (Diovan), beta blockers, such as acebutolol (Sectral), atenolol (Tenormin), betaxolol (Kerlone), bisoprolol/hydrochlorothiazide (Ziac), bisoprolol (Zebeta), carteolol (Cartrol), metoprolol (Lopressor, Toprol XL), nadolol (Corgard), propranolol (Inderal), sotalol (Betapace), timolol (Blocadren), diuretics, such as furosemide (Lasix), bumetanide (Bumex), torsemide (Demadex), hydrochlorothiazide (Esidrix), metolazone (Zaroxolyn), and spironolactone (Aldactone), antiplatelet agents, such as aspirin, ticlopidine, clopidogrel (Plavix), and dipyridamole, cholesterol-lowering drugs, such as statins, resins, nicotinic acid (niacin), gemfibrozil, and clofibrate, and digoxin, and bronchodilators, such as aminophylline, theophylline, salbutamol, salmeterol, bambuterol, clenbuterol, formoterol, indacaterol, tiotropium, and ipratropium bromide, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

Apelin can be administered prior to, concurrent with, or subsequent to other agents. If provided at the same time as other agents, apelin encapsulated in liposomes can be provided in the same or in a different composition. Thus, apelin and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising apelin encapsulated in liposomes and a dose of a pharmaceutical composition comprising at least one other agent, such as another drug for treating a cardiovascular or pulmonary disease or disorder, which in combination comprise a therapeutically effective dose, according to a particular dosing regimen. Similarly, apelin encapsulated in liposomes and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

E. Kits

The invention also provides kits comprising one or more containers holding compositions comprising apelin encapsulated in liposomes, as described herein, or reagents for preparing such compositions, and optionally one or more other drugs for treating a cardiovascular or pulmonary disease or disorder. Compositions can be in liquid form or can be lyophilized, as can separate apelin peptides or liposomes. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating a cardiovascular or pulmonary disease or disorder. The package insert can be an unapproved draft package insert or can be a package insert approved by the Food and Drug Administration (FDA) or other regulatory body.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### [Pyr1]-Apelin-13 Delivery Via Nano-Liposomal Encapsulation Attenuates Pressure Overload-Induced Cardiac Dysfunction

1. Introduction

In this study, a novel liposomal nanocarrier system, incorporated with polyethylene glycol (PEG) polymer on the surface, was utilized in order to deliver [Pyr1]-apelin-13 as a therapeutic molecule into the injury site in a TAC mouse model. The effect of sustained release of [Pyr1]-apelin-13 on the hypertrophic response of the heart was assessed.

2. Materials and Methods

2.1. Liposome Preparation and [Pyr1]-Apelin-13 Encapsulation

Lipids used in the preparation of liposomes include 1,2-distearoyl-sn-glycero-3-phosphoethanolamine-N-[amino (polyethylene glycol)-3400] (DSPE-PEG(3400)-NH₂) (Laysan Bio, Inc.), cholesterol (Sigma), and 1,2-distearoyl-sn-glycero-3-phosphocholine (DSPC) (Avanti polar lipids, Inc.). PEG-containing liposome (lipoPEG) was prepared by dissolving cholesterol (5 mg), NH₂—PEG-DSPE (2 mg), and DSPC (10 mg) in 1 mL of chloroform in a round-bottom (RB) flask. The mixture was evaporated in a rotary evaporator under vacuum to make a thin layer of molecules in the RB flask. The lipid layer was hydrated by the addition of 1 ml double distilled water sonicated for 30 minutes to obtain a turbid liposome solution. The solution was frozen and lyophilized to obtain solvent-free dry liposomes. [Pyr1]-apelin-13 was encapsulated by hydrating the liposomes (17 mg) with 3.3 mg of pyroglutamyl [Pyr1]-apelin-13 in 1 ml double distilled water followed by a 30 minute sonication. In order to get the maximum encapsulation, the mixture was frozen and lyophilized again followed by rehydrating it with 1 mL double distilled water and sonicated before use. Hereby, liposome-PEG-[Pyr1]-apelin-13 (lipoPEG-PA13) was prepared.

2.2. Atomic Force Microscopy

Samples were prepared for atomic force microscopy (AFM) by drop-casting and drying under vacuum of 10 μL droplets with a liposome concentration of 0.01 mg/mL on the surface of clean silicon wafers. AFM imaging was performed with Park Systems NX10 (Suwon, Korea) AFM instrument. Samples were imaged in semi-contact mode with standard commercial cantilevers (k=5-9 N/m, R<10 nm), at 1 Hz scan speed and ˜30% oscillation damping.

2.3. Dynamic Light Scattering Measurements

Solutions were prepared from dry lyophilized liposomes at a concentration of 1 mg/mL. Samples were vortexed until no visible sediment could be detected. Dynamic Light Scattering (DLS) measurements were performed using Brookhaven 90 plus DLS nanosizer (Brookhaven Instruments Corporation, Holtsville, N.Y.). The auto correlation function was approximated manually with a single-relaxation process using second-order term for the cumulant analysis of particle polydispersity.

2.4. In Vitro [Pyr1]-Apelin-13 Release Study

In vitro release of [Pyr1]-apelin-13 was measured using rapid equilibrium dialysis (RED) plate assay (Thermo Scientific Co.). The plate is composed of disposable high-density polypropylene and equilibrium dialysis membrane inserts compartmentalized in to buffer and sample chambers. Each insert is comprised of two side-by-side chambers separated by an O-ring-sealed vertical cylinder of dialysis membrane with molecular weight cutoffs (12 kDa). Stock solution of liposome-PEG-[Pyr1]-apelin-13 (lipoPEG-PA13) was prepared in 2 mL PBS containing 100 nM concentration of [Pyr1]-apelin-13 peptide. The lipoPEG-PA13 sample (750 μL) and the buffer PBS (500 μL) were placed into the corresponding sample (donor) and buffer chambers, respectively. The entire unit was then covered with sealing tape and incubated at 37° C. on an orbital shaker at approximately 300 rpm. At each time point of analysis, a volume of 10 μL was withdrawn from the buffer side of the chamber and replaced with the same volume in return. At 0 hours (start point) and 24 hours (end point), a volume of 10 μL was collected from both the sample (donor) and the buffer chambers. Despite the long duration of incubation time (24 hours), there was no increase in the sample volume due to the hydrostatic pressure. All drug release experiments were done with samples in triplicates. The vials containing collected sample (donor) and buffer solutions were stored at 4° C. before mass spec analysis.

2.5. Liquid Chromatography/Mass Spectrometry Analysis

The concentration of [Pyr1]-apelin-13 released from liposome was measured using liquid chromatography (LC, Shimadzu-LC-20AB Prominence Liquid Chromatogram) coupled with a mass spectrometry (MS) device (4000 Q Trap). Briefly, the collected 10 μL volume of either buffer or donor was added to 20 μL water and 30 μL methanol to precipitate the peptide. Chromatographic separation was performed on a C18 reversed-phase column (XBridge C18, 3.0×150 mm, 3.5 μm particle size) by injecting 10 μL per sample. Gradient elution was employed at a flow rate of 0.3 mL/minute with the following solvent and conditions: 0-0.5 minute 20% acetonitrile/0.1% formic acid, water/0.1% formic acid/5 mM ammonium acetate, 0.5-7 minute gradient from 20%-90%; 7-8 minutes 90%; 8-12 minutes 20%. Based on our established protocol, mass spectrometric detection was conducted specifically at the Q1 (512.060 mass in Da) and Q3 (263.000 mass in Da) regions accomplished with an electrospray ionization (ESI) source in positive ion mode at 5.5 kV spray voltage. Data acquisition was carried out by the AB Sciex Analyst version 1.6.1 software. The concentration of [Pyr1]-apelin-13 in the sample chamber and those released in to the buffer chamber were measured from the calculated peak areas relative to the internal standard.

2.6. In Vitro Assessment of [Pyr1]-Apelin-13 Effect on APJ Translocation

The influence of [Pyr1]-apelin-13 formulation (lipoPEG-PA13)—versus commercially available [Pyr1]-apelin-13 peptide—on the APJ translocation efficacy was tested in vitro, using human embryonic kidney (HEK) 293 cells overexpressing GFP-APJ as described elsewhere (Scimia et al. (2012) Nature 488:394-398). HEK cells, transfected with human APJ, were cultured in DMEM containing 1% penicillin/streptomycin and 10% FBS. Following a 60 minute incubation with lipoPEG-PA13, the GFP-APJ fluorescence was tracked using a high resolution confocal laser scanning microscope (Zeiss LSM510 Meta).

2.7. Mouse Model of TAC-[Pyr1]-Apelin-13 Nanocarrier Administration

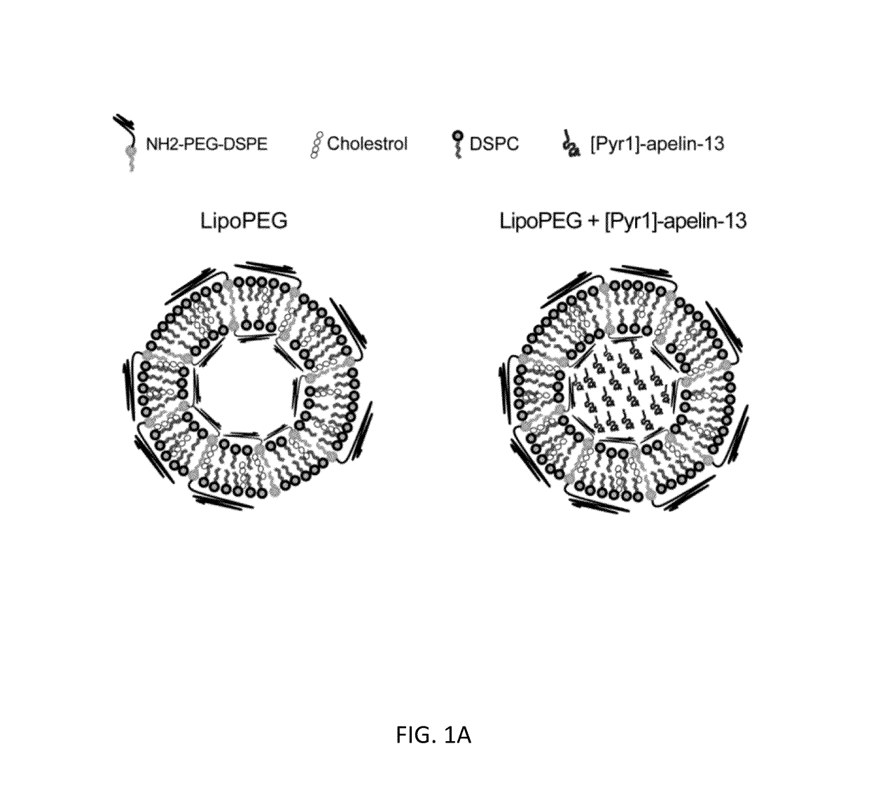

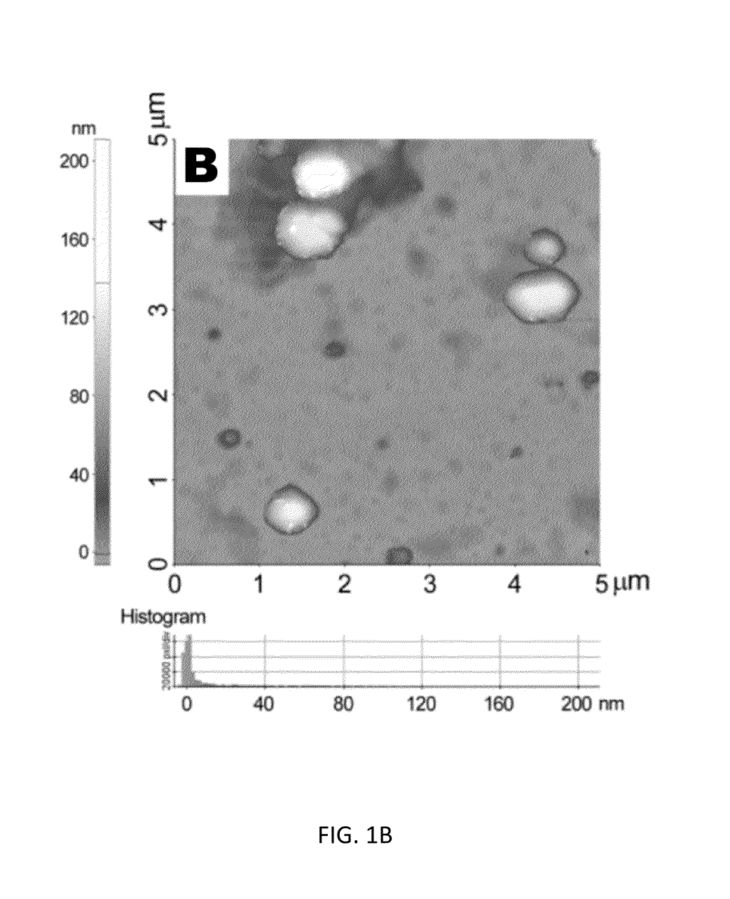

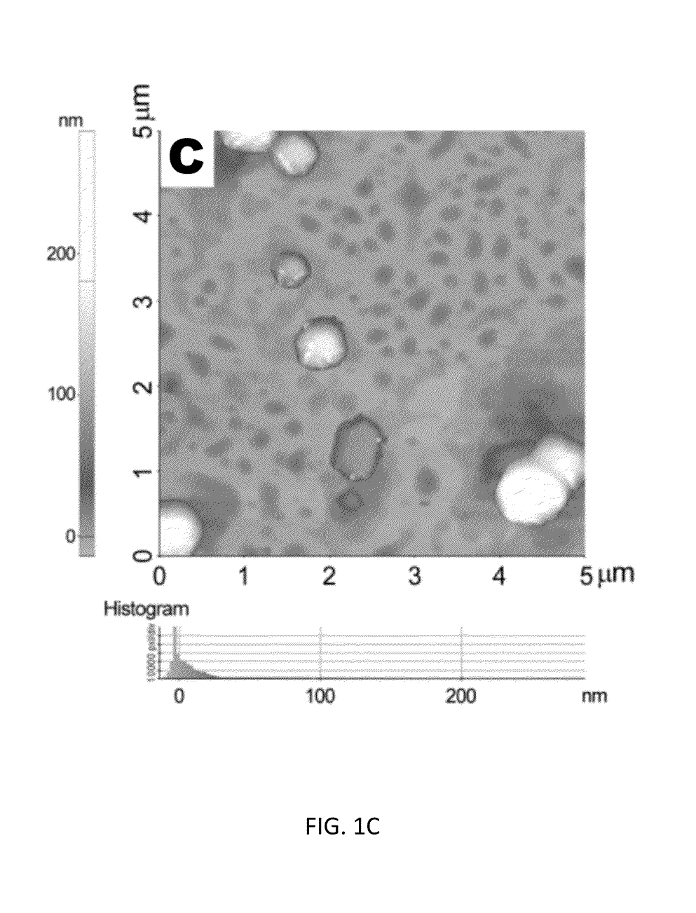

All procedures involving animal use, housing, and surgeries were approved by the Stanford Institutional Animal Care and Use Committee (IACUC). Animal care and interventions were provided in accordance with the Laboratory Animal Welfare Act. Male 10-13 weeks old C57BL/6J mice were purchased from Jackson Laboratories (Bar Harbor, Me., USA) and divided randomly into five groups: I and II) sham controls with no aortic constriction, treated with either saline or [Pyr1]-apelin-13 formulation, III to V) mice with TAC injury treated with either saline, regular [Pyr1]-apelin-13, or lipoPEG-PA13 (n>4, FIGS. 1A-1D).

For constriction of transverse thoracic aorta (DeAlmeida et al. (2010) J. Vis. Exp. April 21(38) pii:172), mice were anesthetized using an isoflurane inhalational chamber, followed by endotracheal intubation using a 22-gauge angiocatheter (Becton, Dickinson Inc., Sandy, Utah) connected to a small animal ventilator (Harvard Apparatus, Holliston, Mass.). A left thoracotomy was performed via the fourth intercostal space, the transverse aorta was identified, and a 6.0 silk suture was placed between the innominate and left carotid arteries. The suture was tightened around a 271/2 gauge blunt needle, placed parallel to the aorta (FIG. 1B) to induce a constriction of ˜0.4 mm in diameter. In sham control mice, an identical procedure was conducted, except for the constriction of the aorta. Mice received [Pyr1]-apelin-13 or saline (control) intraperitoneally (300 μg/kg body weight) at days 1 and 7 post surgery.

2.8. Echocardiography

In vivo heart function was evaluated by echocardiography at 1 day prior to surgery (baseline) and on days 1, 7, and 14 post-surgery (FIG. 1A). Two-dimensional (2D) analysis was performed using a GE Vivid 7 ultrasound platform (GE Health Care, Milwaukee, Wis.) equipped with a 13 MHz transducer. Mice were anesthetized with isoflurane (2% inhalation). 2D clips and M-mode images were obtained in the short axis view from the mid-LV at the tips of the papillary muscles. Fractional shortening (FS), LV internal diameter at end diastole (LVIDd) and systole (LVIDs), and posterior LV wall thickness at end diastole (LVPWd) were measured. A minimum number (n) of 4 mice per study group was used for the echo evaluations. Measurements were performed by two independent observers blinded to study groups.

2.9. Measurement of [Pyr1]-Apelin-13 Concentration in Blood Plasma

24 hours post-surgery, sham and TAC operated mice were injected intraperitoneally with either saline or the [Pyr1]-apelin-13 nanocarriers (lipoPEG-PA13) at 0.4 mg/kg body weight. Wild type mice without surgery and with no injections were used as controls to measure the apelin-13 baseline levels. At days 1, 4, and 6 post injection, mice were anesthetized with isoflurane (100 mg/kg, inhalation) and blood was collected using two different techniques: 1) from the Orbital Sinus (Retro-orbital technique) by inserting the tip of a microhematocrit blood tube into the corner of the eye socket, underneath the eyeball, and directing the tip at towards the middle of the eye socket at a 45-degree angle (details in (Parasuraman et al. (2010) J. Pharmacol. Pharmacother. 1:87-93) from the tail vein; using a straight edge razor, approximately 1 cm of the tail was quickly removed. For additional samples at later time point, blood sample was obtained by removing ˜2-3 mm of additional tail (details in (Parasuraman et al., supra). The blood obtained from either approaches was placed in a polypropylene tube containing 0.1 volume of 3.8% sodium citrate, pH 7.4. Plasma was then prepared by centrifuging the samples at 15,000 g for 10 minutes (at 4° C.) using a table-top microcentrifuge (Eppendorf 5424R). Aliquots were stored at −80° C. before analysis. [Pyr1]-apelin-13 concentrations were determined by commercial enzyme linked immunosorbent assay (ELISA) kits according to the manufacturers' instructions (Apelin EIA kit, Phoenix Pharmaceuticals, Burlingame, Calif.). Each assay was performed in duplicates. Standard curve was fitted using the 4-parameter logistic (4PL) nonlinear regression model (Scimia et al. (2012) Nature 488:394-498).

2.10. Histological Analysis

Histological analysis was performed following standard protocols for paraffin embedding. Mounted heart samples were stained for Masson's trichrome (TC, to label muscle fibers in red, collagen in blue, cytoplasm in pink, and nuclei in dark brown). A minimum of 4 sections per sample were used.

2.11. Statistical Analysis

The number of samples (n) used in each experiment is noted in the text. Dependent variables are expressed as means±SEM, unless noted otherwise. The differences in the means were tested using ANOVA and Student T-test to check for statistical significance (P<0.05).

3. Results

3.1. AFM Characterization of Liposomes and Liposome Encapsulating [Pyr1]-Apelin-13

AFM imaging of the particles, performed in tapping mode, generated the topography maps of the PEG liposomes (FIGS. 1A-1C). Both the empty liposomes and PEG liposomes encapsulating [Pyr1]-apelin-13 exhibited semi-spherical structure with relatively narrow size distribution. The lipoPEG-PA13 nanoparticles formed objects akin to “sunny side up” fried eggs. In addition, the [Pyr1]-apelin-13 loaded particles demonstrated noticeably greater height (˜250 nm) in comparison with that of the empty particles (˜200 nm) (FIGS. 1B and 1C, respectively).

3.2. Particle Size Characterization Using Dynamic Light Scattering

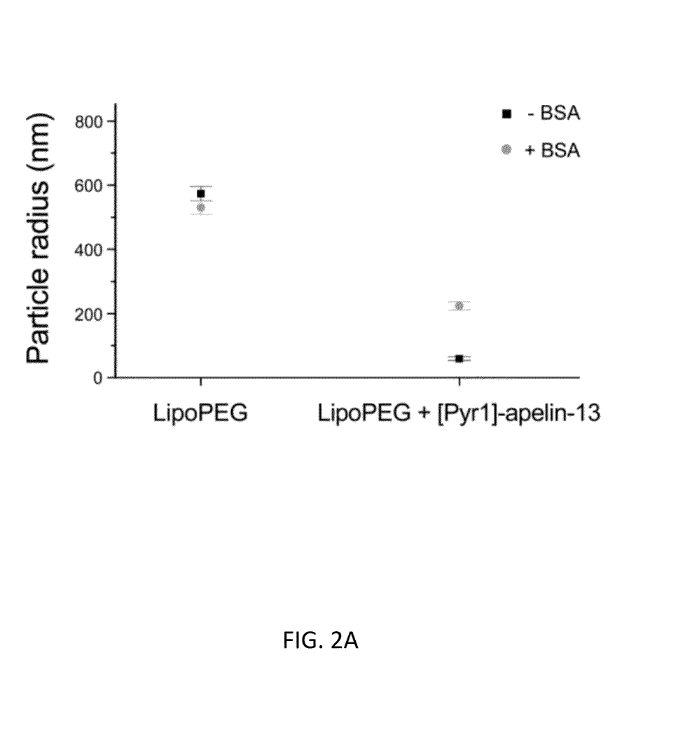

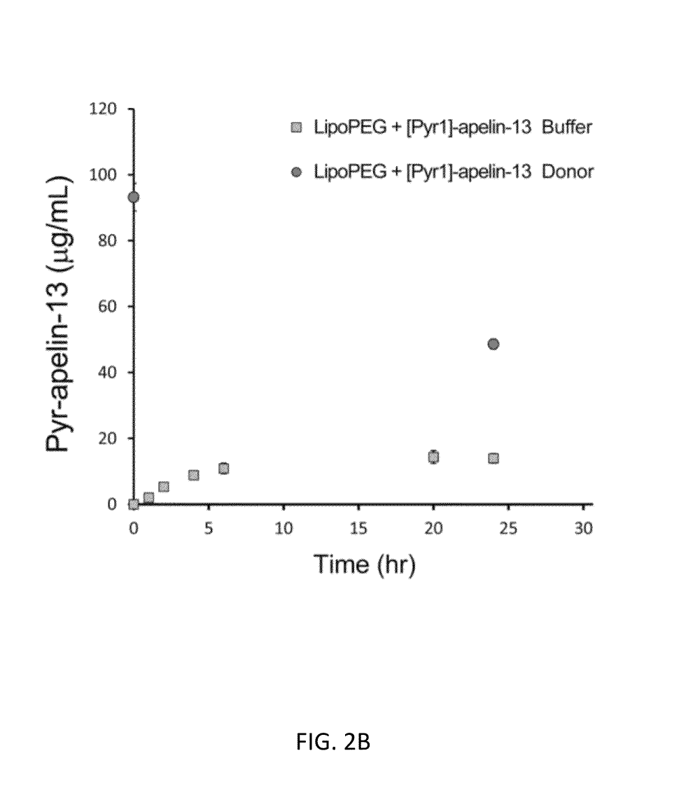

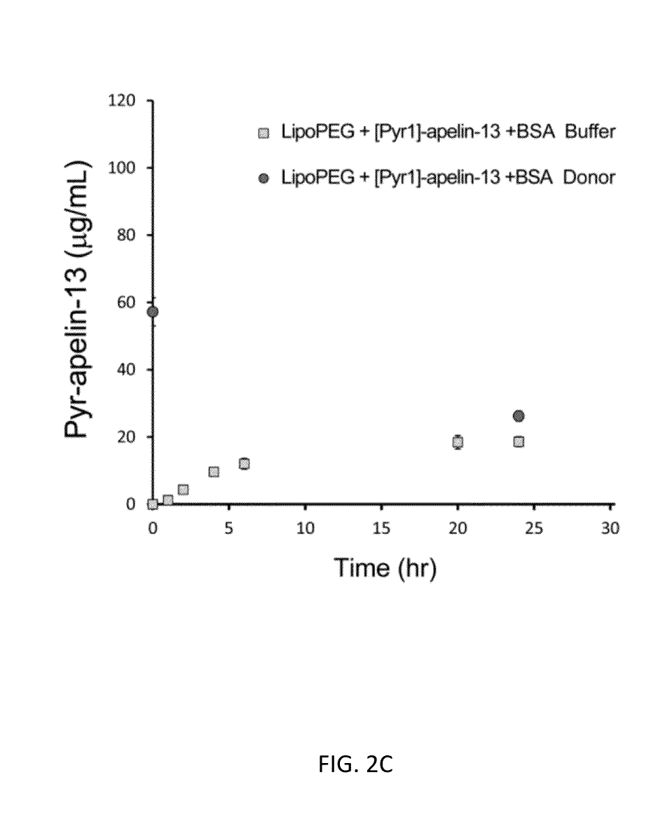

Dynamic light scattering (DLS) was used to analyze the size distribution of the empty lipo-PEG particles and the particles laden with [Pyr1]-apelin-13, in the absence or presence of 0.5% BSA (FIG. 2A). The average size of the lipo-PEG alone was estimated to be 0.57±0.02 (SD) μm. Upon inclusion of 0.5% BSA, there was almost no change in size (0.53±0.02 (SD) μm) for lipo-PEG particles. However, [Pyr1]-apelin-13 encapsulation in lipoPEG showed a striking reduction in particle size to 57±5 nm. Inclusion of 0.5% BSA to particles with encapsulated [Pyr1]-apelin-13 increased their size to 0.22±0.02 (SD) μm.

3.3. Liposomal Encapsulation Promotes Sustained In Vitro Release of [Pyr1]-Apelin-13

[Pyr1]-apelin-13 encapsulation in lipoPEG particles (lipoPEG-PA13) resulted in sustained and extended drug release under physiological conditions in vitro when monitored over a 24 hour period at different time intervals (FIGS. 2B and 2C). The peak [Pyr1]-apelin-13 release profile was obtained between 15 and 20 hour period followed by a saturation curve at the end of 24 hours. [Pyr1]-apelin-13 release from lipoPEG-PA13 was accelerated (18.6 μg/mL) in the presence of 0.5% BSA (FIG. 2C).

3.4. lipoPEG-PA13 Nanocarriers Promote APJ Translocation In Vitro

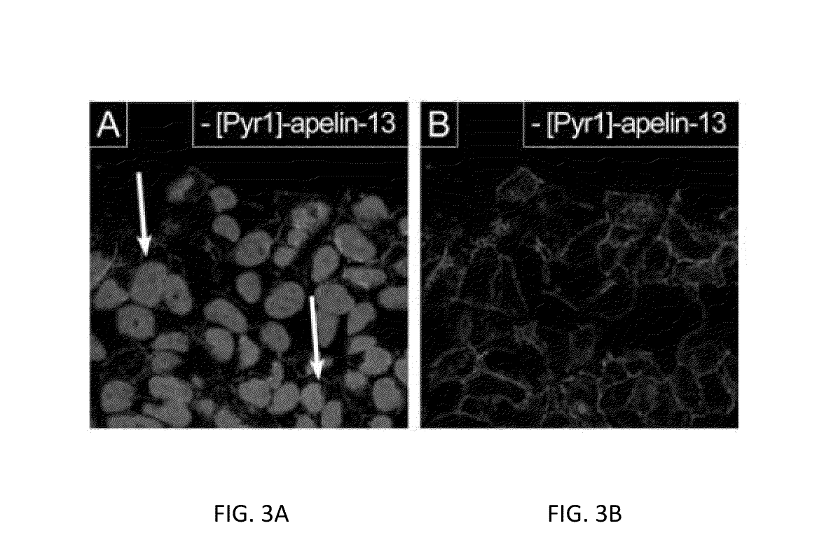

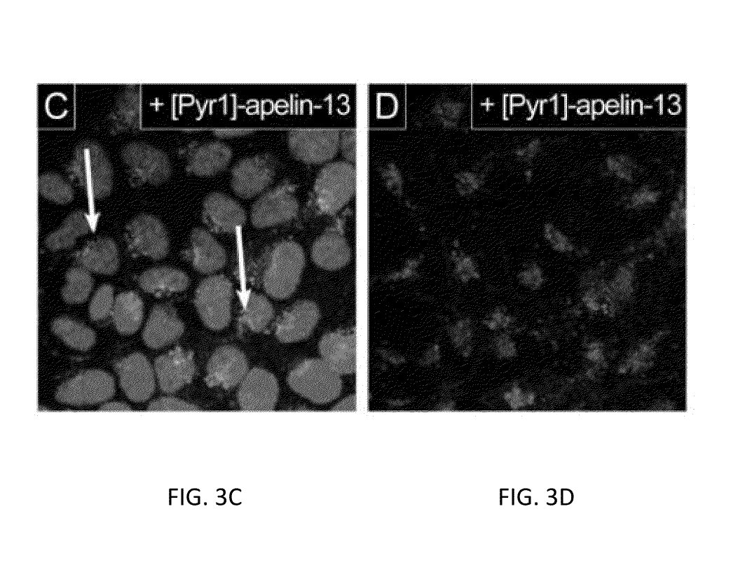

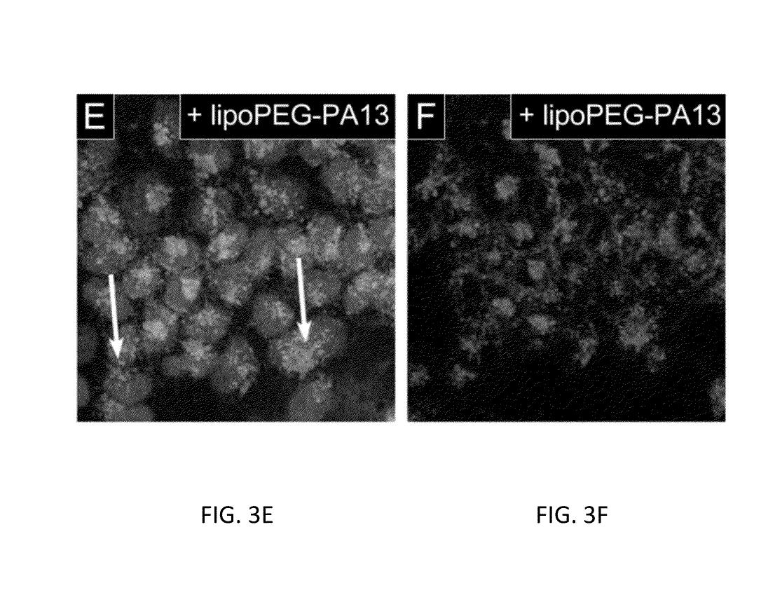

The in vitro effect of [Pyr1]-apelin-13 nanocarriers on APJ translocation was analyzed in APJ-GFP′ HEK293 cells and compared to that induced by commercially available [Pyr1]-apelin-13 (300 nM in culture media (Scimia et al., supra), FIG. 3). Tracing the fluorescent APJ receptors using high-resolution confocal microscopy demonstrated that lipoPEG-PA13 nanocarriers stimulated APJ translocation from the cell membrane towards the cytoplasm more efficiently than that caused by the regular [Pyr1]-apelin-13. The lipoPEG-PA13 nanocarriers also exhibited a qualitatively higher GFP signal after 60 minutes (FIG. 3).

3.5. Treatment with lipoPEG-PA13 Nanocarriers Attenuates TAC-Induced Cardiac Dysfunction In Vivo

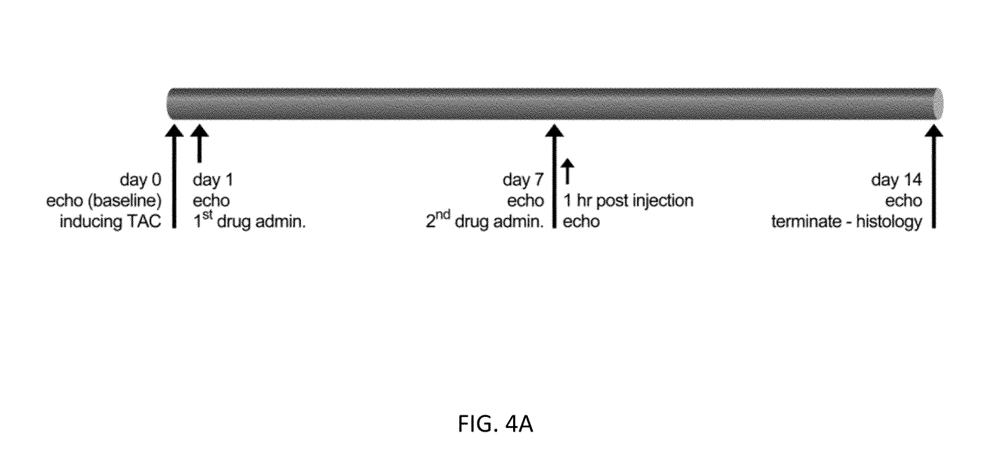

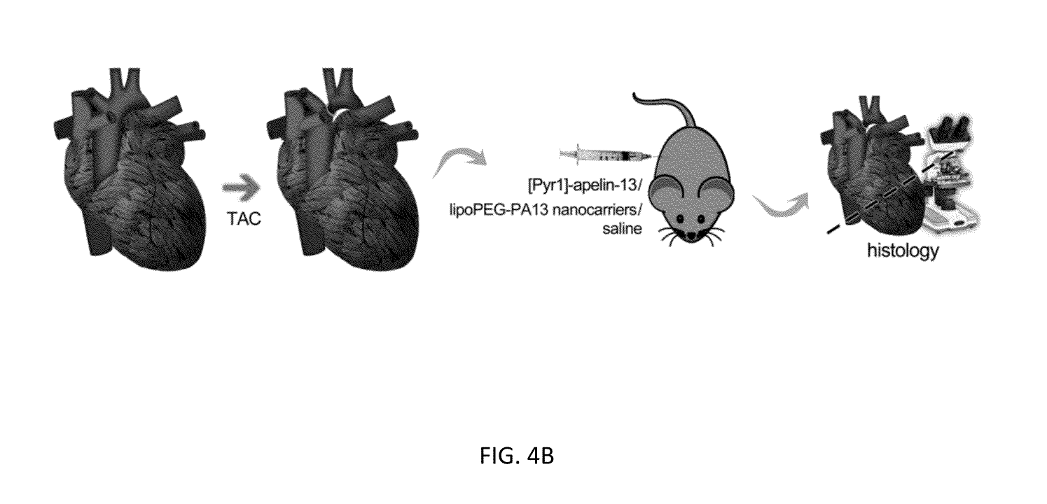

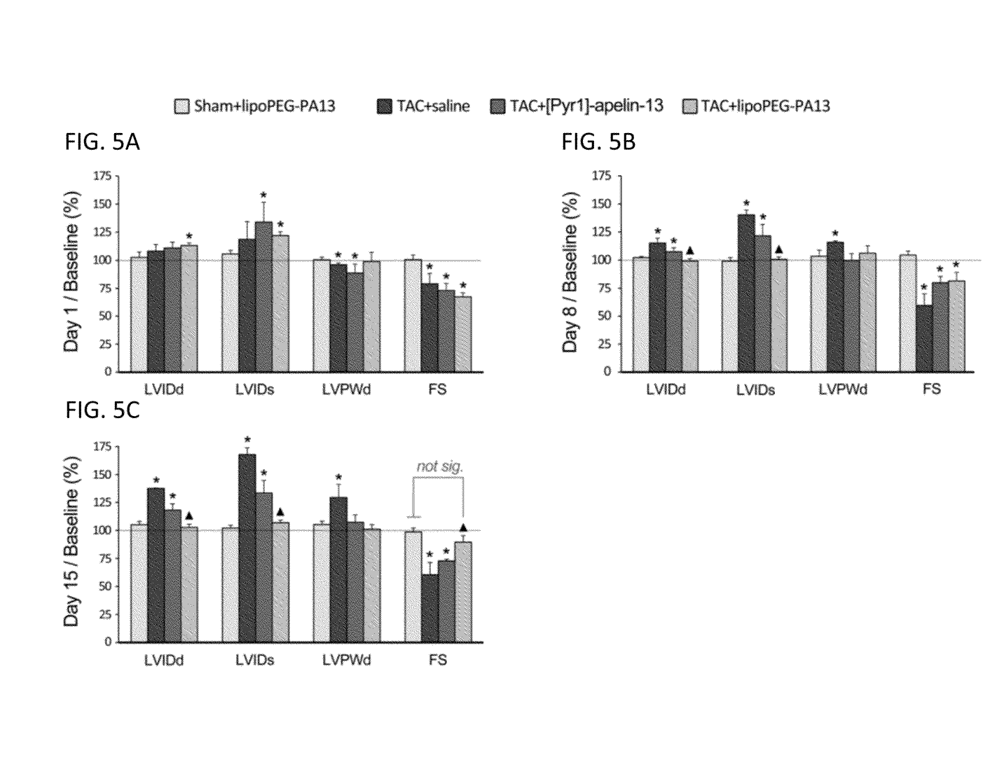

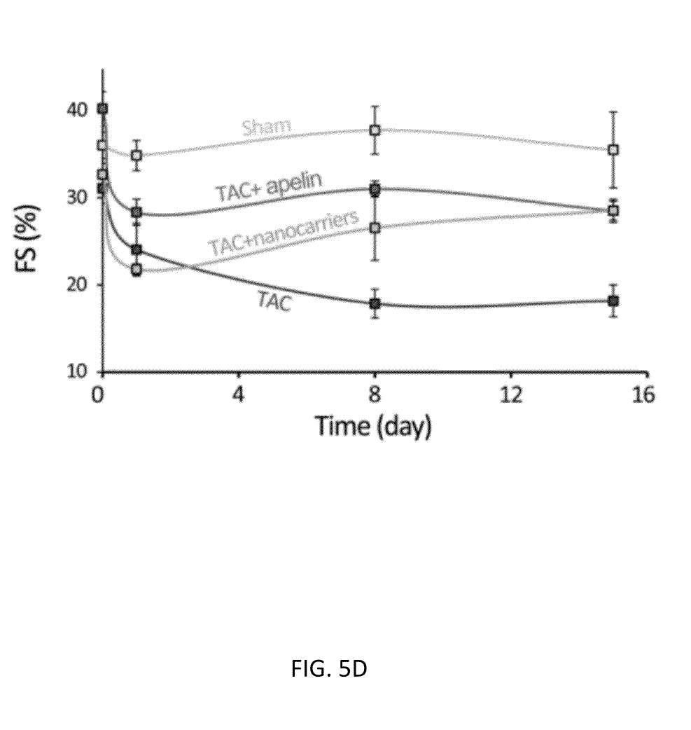

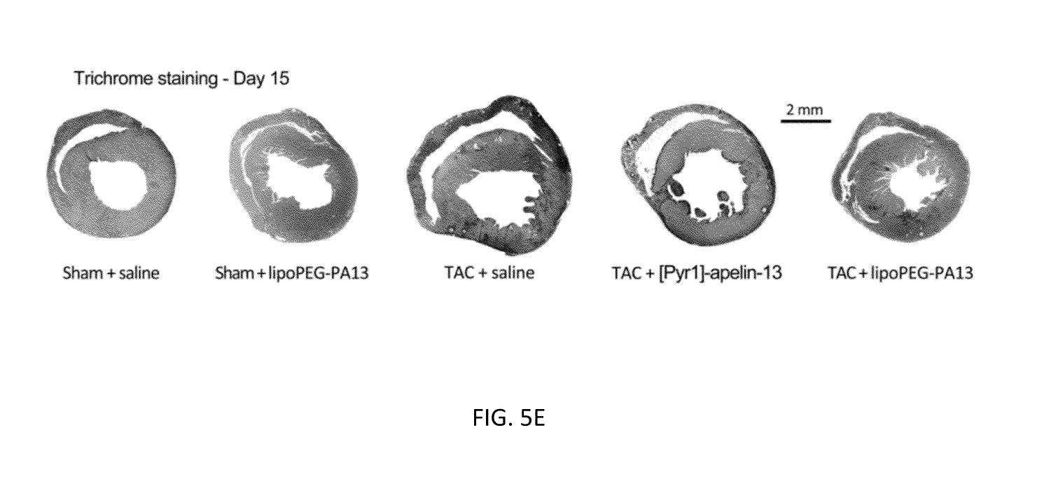

The lipoPEG-PA13 was administered in vivo in TAC operated mice and sham-operated controls by means of single weekly injections for two weeks (FIG. 4). Serial echocardiography at baseline and days 1, 8, and 15 after injury demonstrated significant beneficial effects of lipoPEG-PA13 administration in inhibiting pressure overload-induced LV dysfunction (FIG. 5). No significant differences were observed in echo parameters of the three TAC groups at day 1 post surgery (FIG. 5A). In saline-treated animals (TAC+saline), TAC resulted in LV hypertrophic growth, chamber dilation, and a significant drop in cardiac function at days 8 and 15 post operation (FIGS. 5B and 5C, respectively). In contrast, the application of [Pyr1]-apelin-13 nanocarriers (TAC+lipoPEG-PA13) blunted LV dilation at both end-diastole and systole (LVIDd and LVIDs, respectively), attenuated the increase in posterior wall thickness (LVPWd) at day 15, and preserved cardiac contractility (FS) (FIGS. 5B-5D). In contrast, the administration of commercially available [Pyr1]-apelin-13 (TAC+[Pyr1]-apelin-13) had a more modest effect in diminishing the hypertrophic response to pressure overload. Sham operated groups injected with either saline (data now shown for the simplicity of the graphs) or lipoPEG-PA13 showed no significant changes in cardiac dimensions or function. Thus, lipoPEG-PA13 attenuated pressure overload-induced cardiac dysfunction up to two weeks post injury.

In order to further assess the effect of [Pyr1]-apelin-13 nanocarriers on heart tissue structure and remodeling, mice were sacrificed two weeks post injury and hearts were analyzed. Histology (trichrome staining) of heart sections demonstrated no significant changes in cardiac morphology of sham controls, either in saline or [Pyr1]-apelin-13 treated animals (FIG. 5E). TAC+saline treated mice showed a remarkable increase in the size of LV and the amount of fibrotic tissue. TAC mice treated with lipoPEG-PA13 showed reductions in both the size of the LV and the extent of cardiac fibrosis, when compared to TAC+saline and TAC mice treated with regular [Pyr1]-apelin-13 (FIG. 5E).

3.6. Liposomal Encapsulation Promotes Sustained Release of [Pyr1]-Apelin-13 In Vivo

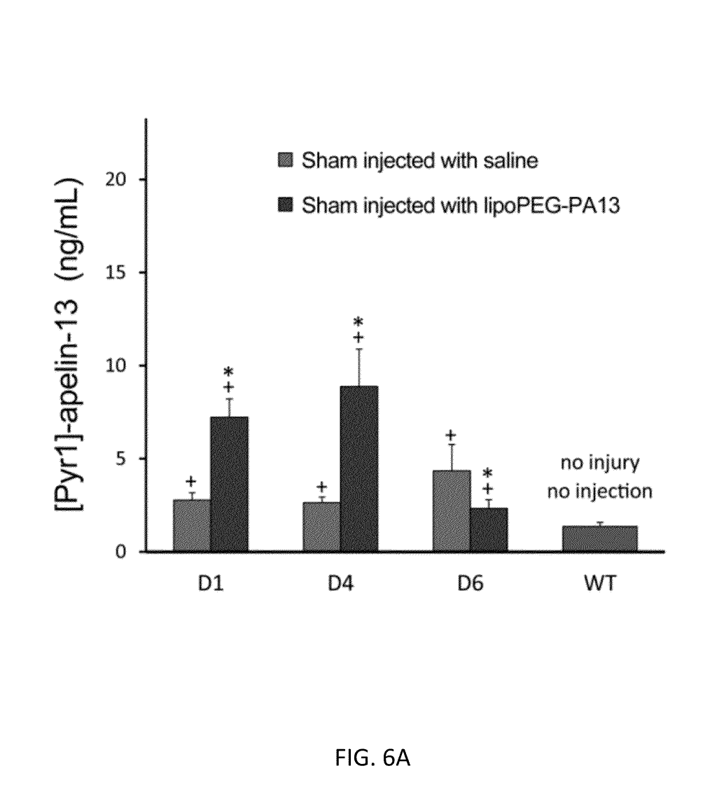

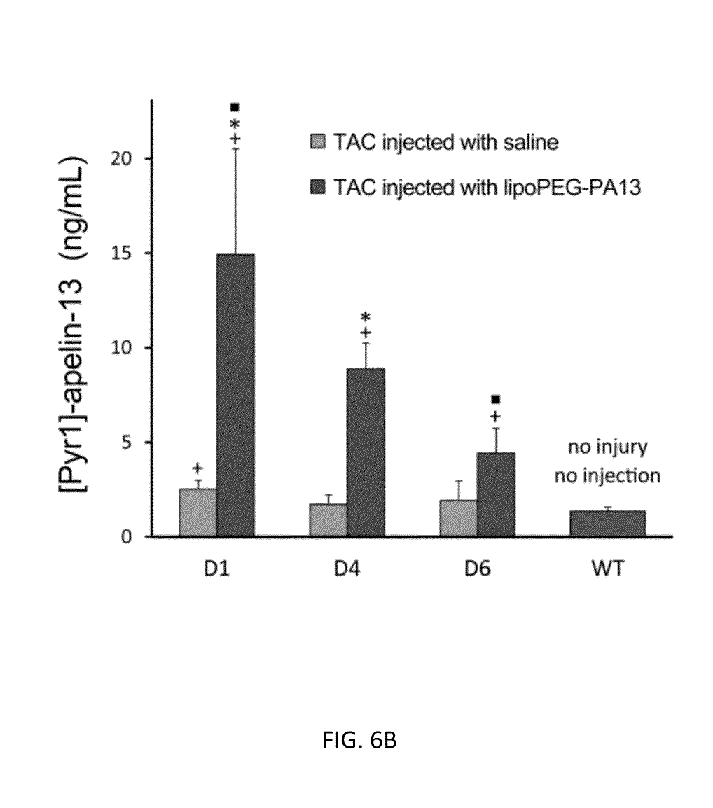

IP administration of lipoPEG-PA13 in both sham-operated and TAC mice resulted in significantly elevated levels of [Pyr1]-apelin-13 in blood plasma at days 1, 4, and 6 post injection, when compared to those in control animals (injected with saline, or not injected, WT) (FIGS. 6A and 6B). The peak [Pyr1]-apelin-13 levels in blood plasma in the TAC and sham mice were obtained at days 1 and 4 post injection, respectively. Moreover, in comparison between sham (▪) and TAC (▪) mice injected with lipoPEG-PA13, [Pyr1]-apelin-13 concentrations in TAC mice were significantly greater than that in sham group at each time point (except day 4).

4. Discussion

Recent studies have shown that the loss of apelin function, together with deficiency of its receptor APJ, correlated with cardiovascular disease (Kuba et al. (2007) Circ. Res. 101:e32-42; Pitkin et al. (2010) Br. J. Pharmacol. 160:1785-1795; Japp et al. (2010) Circulation 121:1818-1827). Some studies have consistently suggested that decreased plasma or myocardial levels of apelin/APJ levels can lead to the incidence of various cardiac defects (Koguchi et al. (2012) Circ. J. Off. J. Jpn. Circ. Soc. 76:137-144; Katugampola et al. (2001) Br. J. Pharmacol. 132:1255-1260; Tasci et al. (2007) Exp. Clin. Endocrinol. Diabetes Off. J. Ger. Soc. Endocrinol. Ger. Diabetes. Assoc. 115:428-432). To date, a significant amount of research has evaluated the role of apelin administration in regulating cardiac dysfunction in various models of heart disease, introducing this peptide as an attractive target for pharmacotherapy studies in the setting of heart failure (Szokodi et al. (2002) Circ. Res. 91:434-440; Ashley et al. (2005) Cardiovasc. Res. 65:73-82; Japp et al. (2010) Circulation 121:1818-1827; Andersen et al. (2011) Pulm. Circ. 1:334-346). Among other beneficial effects, apelin delivery using osmotic pumps has been shown to prevent aortic aneurism (Leeper et al. (2009) Am. J. Physiol. Heart Circ. Physiol. 296:H1329-1335), ameliorate myocardial reperfusion injury (Kleinz et al. (2008) Regul. Pept. 146:271-277), induce vasodilatation (Japp et al. (2008) J. Am. Coll. Cardiol. 52:908-913), and prevent deleterious hypertrophy remodeling (Scimia et al. (2012) Nature 488:394-398). However, the success of conventional systemic drug administration approaches is limited due to several factors including the lack of targeting capability into a pathological site, poor control of sustained drug delivery during the desired therapeutic time, the necessity of a high dose at the target site, nonspecific toxicity, and peptide instability (Mahmoudi et al. (2011) Adv. Drug Deliv. Rev. 63:24-46; Kumar et al. (2013) Biotechnol Adv. 31:593-606; De Jong et al. (2008) Int. J. Nanomedicine 3:133-149). Here, we investigated the use of a liposome nanocarrier system for sustained delivery of exogenous [Pyr1]-apelin-13 and its effect on TAC-induced cardiac hypertrophy in the mouse model.

In order to increase the stability of the nano-liposome in the circulation, the absorbance of the opsonin-based proteins at the surface of nanoparticles should be prevented (Mahmoudi et al. (2011) Chem. Rev. 111:5610-5637). One of the well-recognized approaches for enhancing stability of nano-size materials in circulation is to coat their surfaces with polyethylene glycol (PEG) polymers (Mahon et al. (2012) J. Control Release Off. J. Control Release Soc. 161:164-174). PEG polymer has been used extensively for the covalent alteration of biological macromolecules and surfaces for various biomedical and pharmaceutical applications (Jia et al. (2012) Peptides 38:181-188; Roberts et al. (2002) Adv. Drug Deliv. Rev. 54:459-476). This polymeric shell can prevent plasma's opsonin protein interactions with the surface of nanocarriers and hence rescue their removal by the reticuloendothelial system (Gunaseelan et al. (2010) Adv. Drug Deliv. Rev. 62:518-531; Rezler et al. (2007) Methods Mol. Biol. 386:269-298; Rezler et al. (2007) J. Am. Chem. Soc. 129:4961-72; Chono et al. (2009) J. Control Release Off. J. Control Release Soc. 137:104-109). The PEGylation process increases the molecular mass of protein complexes and shields them against proteolytic enzymes, and hence, prolongs their circulation half-life (Jia et al. (2012) Peptides 38:181-8; Harris J M, Chess RB. Effect of pegylation on pharmaceuticals. Nat Rev Drug Discov 2003; 2:214-221). Thereupon, a lower number of injections or a less chronic drug administration will be required to maintain plasma concentrations of the PEGylated proteins or peptides (Lee et al. (2003) Pharm. Res. 20:818-825). In this study, PEG polymer was incorporated onto the surface of the liposome carriers in order to improve blood plasma stability during in vitro and in vivo experiments.

AFM imaging of the nanocarriers demonstrated the integration of [Pyr1]-apelin-13 into the liposomal particles, with the formation of a mostly homogeneous structure without a clearly defined core and shell (FIG. 1). If we approximate the shape of the two particles in FIG. 1A with a half of an oblate ellipsoid, the volume would be:

$$\frac{{V}_{S}}{S} oblate ellipso id = \frac{1}{2} \cdot \frac{4}{3} π {a}^{2} b \qquad ( Equation 1 )$$

where a and b are halves of long and short axes of the ellipsoid, respectively. The same particles would take a shape of a sphere in solution, the diameter of which would be:

$$D = 2 \cdot {( \frac{v}{4 / {3}^{n}} )}^{1 / 3} \qquad ( Equation 2 )$$

The diameters, obtained in such a fashion are about 0.5 μm, which is in close agreement with the DLS data (FIG. 2A). Using this approximation, the typical volume and, therefore, the solution size of BSA-coated liposomes were calculated to be about the same.

The DLS characterization of plain liposomes (lipo-PEG) and those encapsulated with [Pyr1]-apelin-13 demonstrated relatively high monodispersity of all sets of particles which was not significantly affected by the addition of BSA (FIG. 2A). Interestingly, it appears that the addition of BSA drives the particle size to 0.5 μm, which can be related to the general stability of a BSA-coated particle in a water solution. The relatively large size of particles contributes to the extension of the release period, since it takes longer for the particles to get incorporated through the outer skin layers.

Considering the fact that sustained drug release is an important pharmacokinetic entity, the effect of lipo-PEG (14 μg/mL) particles in sustained and prolonged in vitro release of [Pyr1]-apelin-13 over a 24 hour time course was quite significant (FIGS. 2B and 2C). [Pyr1]-apelin-13 release from lipoPEG-PA13 carriers was accelerated (18.6 μg/mL) in the presence of 0.5% BSA (FIG. 2C) suggesting that BSA had no effect in promoting the drug sustainability. Several studies have reported efficient targeting of protein or peptide based drugs via PEG liposome encapsulation (Visser et al. (2005) Eur. J. Pharm. Sci. Off. J. Eur. Fed. Pharm. Sci. 25:299-305; Xie et al. (2005) J. Control Release Off. J. Control. Release Soc. 105:106-119; Robinson et al. (2002) In Vivo 16(6):535-540). Of particular note, PEGylated DSPE liposomes have shown great potential in promoting the sustained release of several peptide based drugs in vitro (Woodle et al. (1992) Biochim Biophys Acta 1105:193-200; Kim et al. (1999) Int. J. Pharm. 180:75-81; Choi et al. (2011) ACS Nano 5:8591-8599).

Enhanced in vitro efficacy of [Pyr1]-apelin-13 via nano-encapsulation was also demonstrated by significantly increased APJ translocation in the HEK cells when compared to that induced by [Pyr1]-apelin-13 alone (FIG. 3). The cationic PEGylated liposomes are recognized for their intricate ability to become incorporated into pre-formed vesicles, hence, improving cellular binding in vitro (Fenske et al. (2001) Biochim. Biophys. Acta 1512:259-272). Cellular uptake of peptide-encapsulating PEGylated liposomes has been reported to take place through the clathrin-mediated endocytosis pathway (Kibria et al. (2011) J. Control Release Off J. Control Release Soc. 153:141-148).

Administration of lipoPEG-PA13 nanocarriers after TAC demonstrated a significant benefit of the drug in attenuating pressure overload-induced cardiac dysfunction (FIG. 5). On day 8, the administration of lipoPEG nanocarriers resulted in significantly reduced LV dimensions (P<0.05) when compared with those in regular [Pyr1]-apelin-13-treated mice, while there was no significant difference in LV contractility (FS) (FIG. 5B). This suggests that at earlier time points, lipoPEG-PA13 administration effectively interfered with cardiac hypertrophic remodeling. At week 2, in contrast to the TAC mice treated with saline or regular [Pyr1]-apelin-13 (without carriers), administration of the lipoPEG-PA13 resulted in a significant improvement in LV contractility (FS) (P<0.05). We have previously demonstrated that naked [Pyr1]-apelin-13 administration required continuous infusion through osmotic pump to achieve a comparable effect (Scimia et al., supra). Here we found that the lipoPEG-PA13 formulation, administered only once weekly, was capable of attenuating cardiac hypertrophy and the resulting dysfunction for up to two weeks post injury. It should be noted that the four animal groups studied showed varying levels of absolute FS at baseline (FIG. 5D); thus, we utilized percent change from baseline to correct for these differences (FIGS. 5A-5C). Hence, although the absolute FS of the TAC+apelin group was above that of the TAC+nanocarriers group at several time points in FIG. 5D, comparatively; the percentage improvement in contractility was significantly greater in the nanocarrier group, as demonstrated in FIG. 5C. Histology of the mouse hearts treated with lipoPEG-PA13 nanocarriers confirmed the in vivo effect of [Pyr1]-apelin-13 nanocarriers in diminishing fibrosis and cardiac hypertrophy two weeks post injury (FIG. 5E).

The in vivo drug release study performed on the TAC, sham-operated, and wild type (with no surgery) mice revealed the significant effect of the lipo-PEG nano-capsulation in prolonging the apelin-13 lifetime in the blood plasma for up to 6 days (FIG. 6). This is while regular commercial apelin appears rapidly clears from the blood circulation, with a drastically short plasma half-life (no longer than 8 minutes) (Japp et al. (2008) J. Am. Coll. Cardiol. 52:908-913). Remarkably, the effect of nanocarriers in sustained [Pyr1]-apelin-13 release was more significant in the TAC mice, in comparison with that in sham controls. This can be related to the induced changes in plasma proteins due to the acute cardiac (TAC) injury and/or the stress caused by surgery (known as the acute phase response) (Rosenson et al. (1993) J. Am. Coll. Cardiol. 22:933-940; Mackiewicz A, Kushner I, Baumann H. Acute Phase Proteins Molecular Biology, Biochemistry, and Clinical Applications. CRC Press; 1993; Gruys et al. (2005) J. Zhejiang Univ. Sci. B 6:1045-56; Sanchez et al. (2002) Cell Stress Chaperones 7:36-46; Cray et al. (2009) Comp Med 59:517-526). It is well recognized that the surface of nanocarriers gets covered by various proteins upon their entrance into the biological fluid (e.g., blood (Mahmoudi et al. (2011) Chem. Rev. 111:5610-5637; Monopoli et al. (2012) Nat. Nanotechnol. 7:779-786). The type, amount, and thickness of the associated proteins on the surface of nanocarriers (so called “protein corona”) can define their biological fate (Monopoli et al. (2011) J. Am. Chem. Soc. 133:2525-2534). Since the protein source determines the type and composition of associated proteins on the surface of these carriers (Hajipour et al. (2014) Biomater Sci 2(9):1210-1221), the variation of plasma proteins in TAC-operated mice may change the protein corona thickness and composition, hence, leading to a prolonged blood circulation time. Further assessment of the potential role of protein corona formation on the drug release from nanocarriers would be of interest for the future investigations.

5. Conclusions

In summary, we have developed an engineered nanocarrier system, consisting of liposomal nanocarriers incorporating PEG polymer, that demonstrated significantly enhanced efficacy compared to the non-encapsulated, commercially available [Pyr1]-apelin-13, in the delivery and sustained release of drug both in vitro and in vivo. Administration of [Pyr1]-apelin-13 nanocarriers significantly attenuated left ventricular hypertrophy and pressure overload-induced cardiac dysfunction in the mouse model of TAC. An increase in the particles polydispersity could be a further beneficial way to prolong the release. Testing release characteristics of a highly-polydisperse particle formulation would be of interest for the future studies. Further investigations on other therapeutic factors that can be delivered using these carriers, and modification and/or optimization of these devices can establish a new generation of drug delivery systems with unprecedented efficacy to treat various heart injuries and diseases.

Thus, novel compositions and methods for treating cardiovascular and pulmonary diseases with apelin are disclosed. Although preferred embodiments of the subject invention have been described in some detail, it is understood that obvious variations can be made without departing from the spirit and the scope of the invention as described herein.

What is claimed is:

1. A composition comprising an apelin peptide encapsulated in a polyethylene glycol (PEG)-conjugated liposome.

2. The composition of claim 1, wherein the liposome comprises lipids selected from the group consisting of 1,2-distearoyl-sn-glycero-3-phosphoethanolamine-N-[amino (polyethylene glycol)-3400] (DSPE-PEG(3400)-NH₂), cholesterol, and 1,2-distearoyl-sn-glycero-3-phosphocholine (DSPC).

3. The composition of claim 1, wherein the apelin peptide comprises an amino acid sequence selected from the group consisting of SEQ ID NOS:1-7.

4. The composition of claim 1, wherein the apelin peptide comprises a sequence having at least 95% identity to an amino acid sequence selected from the group consisting of SEQ ID NOS:1-7.

5. The composition of claim 4, wherein the apelin peptide comprises a sequence having at least 90% identity to an amino acid sequence selected from the group consisting of SEQ ID NOS:1-7.

6. The composition of claim 5, wherein the apelin peptide comprises a sequence having at least 80% identity to an amino acid sequence selected from the group consisting of SEQ ID NOS:1-7.

7. The composition of claim 6, wherein the apelin peptide comprises a sequence having at least 70% identity to an amino acid sequence selected from the group consisting of SEQ ID NOS:1-7.

8. The composition of claim 1, wherein the apelin peptide is pyroglutamyl apelin-13.

9. The composition of claim 1, wherein the PEG-conjugated liposome comprises PEG-3400.

10. The composition of claim 1, wherein the liposome further comprises bovine serum albumin.

11. The composition of claim 1, wherein the size of the liposome ranges from about 57 nm to about 0.22 μm in diameter.

12. The composition of claim 1, further comprising a pharmaceutically acceptable excipient.

13. The composition of claim 1, further comprising one or more drugs for treating a subject for a cardiovascular or pulmonary disease or disorder.

14. The composition of claim 13, wherein the one or more drugs for treating a subject for a cardiovascular or pulmonary disease or disorder are selected from the group consisting of a vasodilator, an endothelin receptor antagonist, a calcium channel blocker, an anticoagulant, a diuretic, an angiotensin-converting enzyme (ACE) inhibitor, an angiotensin II receptor blocker, a beta blocker, an antiplatelet agent, a cholesterol-lowering drug, and a bronchodilator

15. A method of treating a subject for a cardiovascular or pulmonary disease or disorder, the method comprising administering a therapeutically effective amount of the composition of claim 12 to the subject.

16. The method of claim 15, wherein the composition is administered intravenously, subcutaneously, intralesionally, or intraperitoneally.

17. The method of claim 15, wherein the composition is administered locally into the heart or vascular system.

18. The method of claim 15, wherein the subject is human.

19. The method of claim 15, wherein the subject has hypertrophy, fibrosis, hypertension, heart failure, cardiomyopathy, atherosclerosis, aortic aneurism, myocardial reperfusion injury, or infarction.

20. The method of claim 15, wherein treating the subject induces angiogenesis in the subject.

21. The method of claim 15, wherein treating the subject induces proliferation of myocardial progenitor cells in the subject.

22. The method of claim 15, wherein treating the subject induces vasodilatation in the subject.

23. The method of claim 15, wherein treating the subject attenuates pressure overload-induced cardiac dysfunction.

24. The method of claim 15, wherein treating the subject increases cardiac contractility.

25. The method of claim 15, wherein treating the subject increases cardiac output.

26. The method of claim 15, wherein the composition is administered intermittently.

27. The method of claim 26, wherein the composition is administered once or twice weekly.

28. The method of claim 26, wherein the composition is administered every other week.

29. A method of preparing the composition of claim 2, the method comprising:
a) dissolving cholesterol, NH₂—PEG-DSPE and DSPC in a solvent;
b) evaporating the solvent to produce a thin lipid layer;
c) adding water to the lipid layer;
d) sonicating to produce a turbid liposome solution;
e) lyophilizing the liposome solution to produce dry liposomes; and
f) encapsulating apelin in the liposomes.

30. The method of claim 29, where encapsulating apelin in the liposomes comprises adding an aqueous solution comprising apelin to the dry liposomes to produce a liposome solution and sonicating the liposome solution.

31. The method of claim 30, further comprising lyophilizing the liposome solution to produce dry liposomes, adding water, and sonicating the liposome solution to increase the number of liposomes comprising encapsulated apelin.

32. A pharmaceutical composition prepared according to the method of claim 29.

33. A kit comprising the composition of claim 1 and instructions for treating a cardiovascular disease or a pulmonary disease.

34. The kit of claim 33, further comprising means for delivering said composition to a subject.

35. A kit comprising the pharmaceutical composition of claim 32 and instructions for treating a cardiovascular disease or a pulmonary disease.
